(19) United States
(12) Patent Application Publication
(10) Pub. No.: US 2026/0165669 A1
(43) Pub. Date: Jun. 18, 2026

(54) MEDICAL IMAGE PROCESSING METHODS AND SYSTEMS

(71) Applicant: SHANGHAI UNITED IMAGING HEALTHCARE CO., LTD., Shanghai (CN)

(72) Inventors: Songsong TANG, Shanghai (CN); Yilin LIU, Shanghai (CN); Liuchun HE, Shanghai (CN); Yun DONG, Shanghai (CN); Yifan WU, Shanghai (CN)

(73) Assignee: SHANGHAI UNITED IMAGING HEALTHCARE CO., LTD., Shanghai (CN)

(21) Appl. No.: 19/532,019

(22) Filed: Feb. 6, 2026

Related U.S. Application Data
(63) Continuation of application No. PCT/CN2024/111276, filed on Aug. 9, 2024.

(30) Foreign Application Priority Data
Aug. 10, 2023 (CN) 202311011137.2
Oct. 23, 2023 (CN) 202311377624.0

(51) Int. Cl. A61B 6/00

(52) U.S. Cl. CPC A61B 6/5235; A61B 6/5217; A61B 6/5258

(57) ABSTRACT

Medical image processing methods and systems are provided. The method is implemented on a PET-CT system including a computed tomography (CT) imaging device and a positron emission computed tomography (PET) imaging device. The method may comprise obtaining PET data of a target object within a field of view (FOV) of the PET imaging device. The method may comprise obtaining CT data of the target object within a FOV of the CT imaging device. The method may comprise generating an attenuation image based on a target registration parameter and the CT data, wherein the target registration parameter characterizes a mapping relationship registering the CT coordinate system and the PET coordinate system, and the target registration parameter is obtained based on background coincidence event data from the PET imaging device. The method may further comprise reconstructing a target PET image based on the PET data and the attenuation image.

### CROSS-REFERENCE TO RELATED APPLICATIONS

This application is a continuation of International Application No. PCT/CN2024/111276, filed on Aug. 9, 2024, which claims priority to Chinese Patent Application No. 202311011137.2, filed on Aug. 10, 2023, and Chinese Patent Application No. 202311377624.0, filed Oct. 23, 2023, the entire contents of each of which are hereby incorporated by reference.

### TECHNICAL FIELD

The present disclosure relates to the field of medical technology, and in particular, to methods and systems for processing medical images.

### BACKGROUND

Positron Emission Computed Tomography-Computer Tomography (PET-CT) imaging can obtain PET images with functional information and CT images with fine anatomical structure information. By fusing PET images and CT images, it is possible to pinpoint the location of lesions and improve the accuracy of disease diagnosis. In PET-CT imaging, PET data needs to be corrected to obtain quantitatively accurate and artifact-free PET reconstructed images. The correction includes attenuation correction, normalization correction, etc. Existing correction manners have various issues, such as being complicated to operate, requiring high standards for personnel and equipment, posing health risks to operators, affecting imaging quality, etc.

For example, in the attenuation correction, a CT image is typically converted and registered to obtain an attenuation image. The registration process requires an operator (e.g., a service engineer, a technician, a physician, etc.) to create a phantom (e.g., a water phantom, etc.) containing a radiation source or use a solid registration source. The phantom is scanned using both CT and PET imaging devices and the corresponding images are reconstructed. Subsequently, the CT reconstructed image and the PET reconstructed image are registered to obtain a mapping relationship between a CT image reconstructed coordinate system and a PET image reconstructed coordinate system. The process of creating a phantom containing a radiation source is time-consuming, labor-intensive, inefficient, and costly. Moreover, the radiation source poses health risks to the operators. Additionally, the registration process demands high standards for both the operators and the PET imaging device. For example, the registration process requires experienced operators.

As another example, during the normalization correction, due to changes in the system state, normalization correction factors need to be updated regularly. If the update cycle is too long, the mismatch between the normalization correction factors and the system state may gradually increase, making it challenging to ensure the effectiveness of the normalization correction and leading to a decline in the quality of the scanned image.

Therefore, it is desired to provide a method and a medical image processing system to improve the efficiency and quality of PET data correction.

### SUMMARY

An aspect of the present disclosure relates to a method for medical image processing. The method may be performed by a computing device including at least one processor and at least one storage device, wherein the method is implemented on a PET-CT system, the PET-CT system includes a computed tomography (CT) imaging device and a positron emission computed tomography (PET) imaging device. The method may include obtaining PET data of a target object within a field of view (FOV) of the PET imaging device. The method may include obtaining CT data of the target object within a FOV of the CT imaging device. The method may include generating an attenuation image based on a target registration parameter and the CT data. The target registration parameter characterizes a mapping relationship registering the CT coordinate system and the PET coordinate system, and the target registration parameter is obtained based on background coincidence event data from the PET imaging device. The method may further include reconstructing a target PET image based on the PET data and the attenuation image.

Another aspect of the present disclosure relates to a system. The system may include at least one storage device including a set of instructions and at least one processor in communication with the at least one storage device. When executing the set of instructions, the at least one processor may be directed to cause the system to implement operations on a PET-CT system, the PET-CT system includes a computed tomography (CT) imaging device and a positron emission computed tomography (PET) imaging device. The operations may include obtaining PET data of a target object within a field of view (FOV) of the PET imaging device. The operations may include obtaining CT data of the target object within a FOV of the CT imaging device. The operations may include generating an attenuation image based on a target registration parameter and the CT data. The target registration parameter characterizes a mapping relationship registering the CT coordinate system and the PET coordinate system, and the target registration parameter is obtained based on background coincidence event data from the PET imaging device. The operations may further include reconstructing a target PET image based on the PET data and the attenuation image.

A further aspect of the present disclosure relates to a non-transitory computer readable medium including executable instructions. When the executable instructions are executed by at least one processor, the executable instructions may direct the at least one processor to perform a method on a PET-CT system, the PET-CT system includes a computed tomography (CT) imaging device and a positron emission computed tomography (PET) imaging device. The method may include obtaining PET data of a target object within a field of view (FOV) of the PET imaging device. The method may include obtaining CT data of the target object within a FOV of the CT imaging device. The method may include generating an attenuation image based on a target registration parameter and the CT data. The target registration parameter characterizes a mapping relationship registering the CT coordinate system and the PET coordinate system, and the target registration parameter is obtained based on background coincidence event data from the PET imaging device. The method may further include reconstructing a target PET image based on the PET data and the attenuation image.

A still further aspect of the present disclosure relates to a method for medical image processing. The method may be performed by a computing device including at least one processor and at least one storage device. The method may include obtaining PET data of a target object within a field of view (FOV) of a positron emission computed tomography (PET) imaging device. The method may include obtaining corrected PET data by correcting the PET data based on a target normalization correction factor. The target normalization correction factor is a normalization correction factor corresponding to a current state of the PET imaging device. The target normalization correction factor is determined by: acquiring first background coincidence event data of a detector crystal of the PET imaging device at a predetermined time point; determining the target normalization correction factor based on the first background coincidence event data; and reconstructing a target PET image based on the corrected PET data.

A still further aspect of the present disclosure relates to a system. The system may include at least one storage device including a set of instructions and at least one processor in communication with the at least one storage device. When executing the set of instructions, the at least one processor may be directed to cause the system to implement operations. The operations may include obtaining PET data of a target object within a field of view (FOV) of a positron emission computed tomography (PET) imaging device. The operations may include obtaining corrected PET data by correcting the PET data based on a target normalization correction factor. The target normalization correction factor is a normalization correction factor corresponding to a current state of the PET imaging device. The target normalization correction factor is determined by: acquiring first background coincidence event data of a detector crystal of the PET imaging device at a predetermined time point; determining the target normalization correction factor based on the first background coincidence event data; and reconstructing a target PET image based on the corrected PET data.

A still further aspect of the present disclosure relates to a method for medical image processing. The method may be performed by a computing device including at least one processor and at least one storage device, wherein the method is implemented on a PET-CT system, the PET-CT system includes a computed tomography (CT) imaging device and a positron emission computed tomography (PET) imaging device. The method may include obtaining a CT image of a phantom by placing the phantom in the FOV of the CT imaging device, the phantom not containing a radiation source. The method may include collecting background coincidence event data of the phantom by placing the phantom in the FOV of the PET imaging device. The method may include acquiring a first attenuation image in a coordinate system of the CT imaging device based on the CT image. The method may further include determining a target registration parameter based on target background coincidence event data, and the first attenuation image. The target background coincidence event data is obtained based on the background coincidence event data of the phantom.

A still further aspect of the present disclosure relates to a method for medical image processing. The method may be performed by a computing device including at least one processor and at least one storage device, wherein the method is implemented on a PET-CT system, the PET-CT system includes a computed tomography (CT) imaging device and a positron emission computed tomography (PET) imaging device. The method may include acquiring first background coincidence event data of a detector crystal of the PET imaging device at a predetermined time point. The method may include obtaining a first background energy response based on the first background coincidence event data. The method may include determining a target normalization correction factor based on the first background energy response.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary medical image processing system according to some embodiments of the present disclosure;

FIG. 2 is a block diagram illustrating an exemplary processing device according to some embodiments of the present disclosure;

FIG. 3 is a flowchart illustrating an exemplary process for processing a medical image according to some embodiments of the present disclosure;

FIG. 4 is a flowchart illustrating an exemplary process for obtaining a target registration parameter according to some embodiments shown in the present disclosure;

FIG. 5 is a flowchart illustrating an exemplary process for determining a target normalization correction factor according to some embodiments of the present disclosure;

FIG. 6 is a flowchart illustrating an exemplary process for determining a relationship between a variation of a background energy response and a variation of a normalization correction factor under different states of a PET imaging device according to some embodiments of the present disclosure;

FIG. 7 is a flowchart illustrating an exemplary process for obtaining a background energy response based on background coincidence event data according to some embodiments of the present disclosure;

FIG. 8 is a schematic diagram illustrating an exemplary system state changing over time according to some embodiments of the present disclosure;

FIG. 9 is a schematic diagram illustrating an exemplary decay energy level of Lu-176 according to some embodiments of the present disclosure;

FIG. 10 is a schematic diagram illustrating an exemplary discrimination process of a background coincidence event in a PET imaging device according to some embodiments in the present disclosure;

FIG. 11A is a schematic diagram illustrating an exemplary background energy response according to some embodiments of the present disclosure;

FIG. 11B is a schematic diagram illustrating an exemplary change in energy response due to a change in a system state according to some embodiments of the present disclosure;

FIG. 12 is a schematic diagram illustrating an exemplary process for obtaining energy responses and normalization correction factors under different system states of a PET imaging device according to some embodiments of the present disclosure;

FIG. 13 is a schematic diagram illustrating an exemplary process for performing normalization correction on clinical scan data according to some embodiments of the present disclosure;

FIG. 14 is a schematic diagram illustrating an exemplary process for obtaining a background energy response according to some embodiments of the present disclosure;

FIG. 15 is a schematic diagram illustrating an exemplary pyramidal tract with a particular crystal as a tip according to some embodiments of the present disclosure;

FIG. 16A is a schematic diagram illustrating an exemplary distribution of a crystal detection efficiency correction factor under a reference system state according to some embodiments of the present disclosure;

FIG. 16B is a schematic diagram illustrating an exemplary distribution of a position of a 307 keV energy peak under a reference system state according to some embodiments of the present disclosure;

FIG. 17A is a schematic diagram illustrating an exemplary distribution of a crystal detection efficiency correction factor after changing a system state according to some embodiments of the present disclosure;

FIG. 17B is a schematic diagram illustrating an exemplary distribution of a position of a 307 keV energy peak after changing a system state according to some embodiments of the present disclosure;

FIG. 18A is a schematic diagram illustrating an exemplary distribution of a variation of a crystal detection efficiency correction factor according to some embodiments of the present disclosure;

FIG. 18B is a schematic diagram illustrating an exemplary distribution of a variation of a position of a 307 keV energy peak according to some embodiments of the present disclosure; and

FIG. 19 is a schematic diagram illustrating an exemplary relationship between a variation of a crystal detection efficiency correction factor and a variation of a position of a 307 keV energy peak according to some embodiments of the present disclosure.

### DETAILED DESCRIPTION

In the following detailed description, numerous specific details may be set forth by way of examples in order to provide a thorough understanding of the relevant disclosure. However, it should be apparent to those skilled in the art that the present disclosure may be practiced without such details. In other instances, well-known methods, procedures, systems, components, and/or circuitry have been described at a relatively high-level, without detail, in order to avoid unnecessarily obscuring aspects of the present disclosure. Various modifications to the disclosed embodiments may be readily apparent to those skilled in the art, and the general principles defined herein may be applied to other embodiments and applications without departing from the spirit and scope of the present disclosure. Thus, the present disclosure may be not limited to the embodiments shown, but to be accorded the widest scope consistent with the claims.

The terminology used herein may be for the purpose of describing particular example embodiments only and may be not intended to be limiting. As used herein, the singular forms “a,” “an,” and “the” may be intended to include the plural forms as well, unless the context clearly indicates otherwise. It may be understood that the terms “system,” “unit,” “module,” and/or “block” used herein are one method to distinguish different components, elements, parts, sections, or assemblies of different levels in ascending order. However, the terms may be displaced by another expression if they achieve the same purpose.

The modules (or units, blocks, units) described in the present disclosure may be implemented as software and/or hardware modules and may be stored in any type of non-transitory computer-readable medium or other storage devices. In some embodiments, a software module may be compiled and linked into an executable program. It may be appreciated that software modules may be callable from other modules or from themselves, and/or may be invoked in response to detected events or interrupts. Software modules configured for execution on computing devices may be provided on a computer-readable medium or as a digital download (and can be originally stored in a compressed or installable format that requires installation, decompression, or decryption prior to execution). Such software code may be stored, partially or fully, on a memory device of the executing computing device, for execution by the computing device. Software instructions may be embedded in a firmware, such as an EPROM. It may be further appreciated that hardware modules (e.g., circuits) may be included in connected or coupled logic units, such as gates and flip-flops, and/or may be included in programmable units, such as programmable gate arrays or processors. The modules or computing device functionality described herein may be preferably implemented as hardware modules, but may be software modules as well. In general, the modules described herein refer to logical modules that may be combined with other modules or divided into units despite their physical organization or storage.

Certain terminology has been used to describe embodiments of the present disclosure. For example, the terms “one embodiment,” “an embodiment,” and/or “some embodiments” may mean that a particular feature, structure, or characteristic described in connection with the embodiment is in at least one embodiment of the present disclosure. Therefore, it is emphasized and should be appreciated that two or more references to “an embodiment,” “one embodiment” or “an alternative embodiment” in various portions of the present disclosure may not necessarily all referring to the same embodiment. Furthermore, the particular features, structures, or characteristics may be combined as suitable in one or more embodiments of the present disclosure.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of this disclosure. It is to be expressly understood, however, that the drawings may be for the purpose of illustration and description only and may be not intended to limit the scope of the present disclosure.

The flowcharts used in the present disclosure may illustrate operations that systems implement according to some embodiments of the present disclosure. It is to be expressly understood, that the operations of the flowcharts may be implemented not in order. Conversely, the operations may be implemented in inverted order, or simultaneously. Moreover, one or more other operations may be added to the flowcharts. One or more operations may be removed from the flowcharts.

In PET-CT systems, correction of PET data is required. The correction includes attenuation correction, normalization correction, etc.

Attenuation correction is usually performed based on an attenuation image. The attenuation image is typically obtained based on a registration parameter through conversion and registration of a CT image. Currently, the registration process in the attenuation correction typically requires an operator to either pour a radiation source into a phantom (e.g., a water phantom, etc.) or use a solid registration source. Images are reconstructed based on scan data of the phantom by a CT imaging device and a PET imaging device, and then the CT reconstructed image and the PET reconstructed image are aligned to obtain a mapping relationship (i.e., the registration parameter) between a CT reconstructed coordinate system and a PET reconstructed coordinate system.

For a system without a CT imaging device, such as a single PET system or a positron emission tomography-magnetic resonance (PET-MR) system, it is not possible to obtain the attenuation image based on the CT image. For a PET system without an external radiation source, it is not possible to obtain the attenuation image based on the external radiation source. For a medical imaging scenario (e.g., medical imaging of children, etc.) that is sensitive to radiation dose, radiation should be minimized by avoiding using the CT or external radiation source. For a PET-CT system, if the quality of the CT image is poor, such as strong artifacts or partial absence, the quality of the attenuation image obtained based on the CT image is poor, which may lead to artifacts or inaccuracies in the PET reconstructed image. In addition, the above registration process requires an experienced operator (e.g., a service engineer, a technician, a doctor, etc.), which is time-consuming and may pose a radiological hazard to the operator.

Currently, most PET systems use lutetium orthosilicate (LSO) or lutetium yttrium orthosilicate (LYSO) crystals, which may produce spontaneous background radiation. For example, a PET system with along axial field of view uses a much larger number of crystals than a PET system with a normal axial field of view, therefore the PET system with the long axial field of view generates a much larger signal intensity of background radiation than the PET system with the normal axial field of view.

Some embodiments of the present disclosure provide a method for processing a medical image, which is implemented in a PET-CT system including a CT imaging device and a PET imaging device. The method includes obtaining PET data of a target object within a field of view (FOV) of the PET imaging device and CT data of the target object within a FOV of the CT imaging device. The method includes obtaining an attenuation image based on a target registration parameter and the CT data. The target registration parameter characterizes a mapping relationship registering the CT coordinate system and the PET coordinate system. The target registration parameter is obtained based on background coincidence event data (also known as spontaneous background radiation data) from the PET imaging device. The method further includes reconstructing a target PET image based on the PET data and the attenuation image.

The present disclosure provides the method for processing the medical image, in which the attenuation image of the target object is obtained by utilizing the spontaneous background radiation of the PET imaging device to obtain the registration parameter. The method of the present disclosure does not require perfusion of the phantom or preparation of a solid registration source, which simplifies the operation process, saves manpower and material resources, improves registration efficiency, and establishes a solid foundation for rapid registration of the PET image and the CT image. The method of the present disclosure may reduce the radiation injury of the radiation source to the operator. Further, by eliminating the need to perfuse the phantom or prepare the solid registration source, the method of the present disclosure may significantly reduce requirements for the hardware of the PET-CT imaging device, and may be suitable for the PET-CT imaging device without an external source mounting device and motion device. Furthermore, in the method of the present disclosure, the attenuation correction is performed without increasing any hardware cost, has good applicability and is easy to deploy and implement. In addition, the method of the present disclosure may be automatically performed outside of clinical scanning time (e.g., during non-working hours such as midnight), ensuring that normal clinical use is not interrupted.

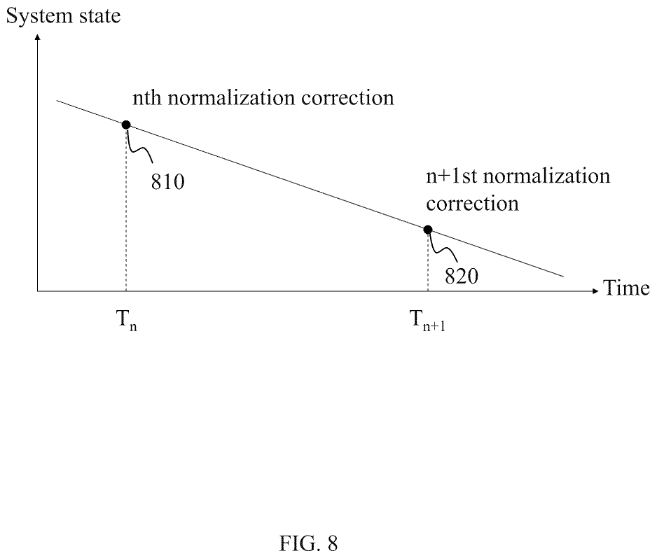

The normalization correction is typically performed based on a normalization correction factor. The normalization correction factor is typically obtained by making the water phantom or scanning with a solid source and processing the scan data. However, this process requires the radiation source and the experienced operator and is time-consuming, takes time away from clinical scanning, and has radiation damage to the operator. In addition, the normalization correction factor needs to be updated periodically due to changes in a system state, e.g., changes in the external environment, aging of the crystal, changes in the Silicon Photomultiplier (SiPM) gain, and changes in the light-harvesting performance from the crystal to the SiPM. If an update period for the normalization correction factor is too long, a mismatch between the normalization correction factor and the system state may increase over time, which may result in a degradation of the quality of a clinical scan image. If the update period of the normalization correction factor is too short, the cost and the radiation dose to related personnel (patients and operators) may be increased. FIG. 8 is a schematic diagram illustrating an exemplary system state changing over time according to some embodiments of the present disclosure. As shown in FIG. 8, the horizontal coordinate denotes time, the vertical coordinate denotes the system state, and the diagonal line in the coordinate system denotes the change in the system state over time. Point 810 denotes the nth normalization correction, which is performed at the moment Tn, and point 820 denotes the n+1st normalization correction, which is performed at the moment Tn+1. Starting from time Tn, the mismatch between the normalization correction factor and the system state gradually increases until the next normalization correction (i.e., the n+1st normalization correction corresponding to time Tn+1).

Usually, if the system state changes, in addition to the scan data being affected, background radiation data is also affected. Thus, a correlation between the background radiation data and the change in the system state may be exploited to predict the change in the normalization correction factor by monitoring the background radiation data. For example, based on the background radiation data, whether the change in the system state has occurred may be determined. If the system state changes, the background radiation data are used to determine the normalization correction factor under the background energy, and the normalization correction factor under the background energy is converted, by a preset conversion factor, to a normalization correction factor under a scanning annihilation radiation energy (511 keV). However, due to a non-linear relationship between changes in a response line and detection efficiency of a detector crystal under irradiation of gamma photons with different energies, using the conversion factor for the conversion of normalization correction factors at different energies may result in significant errors.

The present disclosure provides a method for processing the medical image. The method includes obtaining PET data of a target object within a FOV of a positron emission computed tomography (PET) imaging device and obtaining corrected PET data by correcting the PET data based on a target normalization correction factor. The target normalization correction factor is a normalization correction factor corresponding to a current state of the PET imaging device, and the target normalization correction factor is determined by the following process. First background coincidence event data of the detector crystal of the PET imaging device are acquired at a predetermined time point. A first background energy response is obtained based on the first background coincidence event data. A variation of the first background energy response is generated based on the first background energy response and a first reference background energy response, and the first reference background energy response is obtained under a first reference system state of the PET imaging device. Then, in response to determining that the variation of the first background energy response is not greater than a variation threshold, a first reference normalization correction factor is designated as the target normalization correction factor, and the first reference normalization correction factor is obtained based on a phantom under the first reference system state of the PET imaging device. In response to determining that the variation of the first background energy response is greater than the variation threshold, a variation of a first normalization correction factor is generated based on the variation of the first background energy response and a predetermined relationship, and the target normalization correction factor is determined based on the variation of the first normalization correction factor and the first reference normalization correction factor. In the method of the present disclosure, the normalization correction factor is determined without the need for a radiation source and a CT imaging device, thereby simplifying the operational process of the correction, reducing the requirements for the operator and the devices, and decreasing the radiation damage to the operator from the radiation source, as well as the radiation damage to the patient from the CT imaging device and the external radiation source in the clinical setting. In addition, the method of the present disclosure may be automatically performed outside of clinical scanning time (e.g., during non-working hours such as midnight), ensuring that normal clinical use is not interrupted.

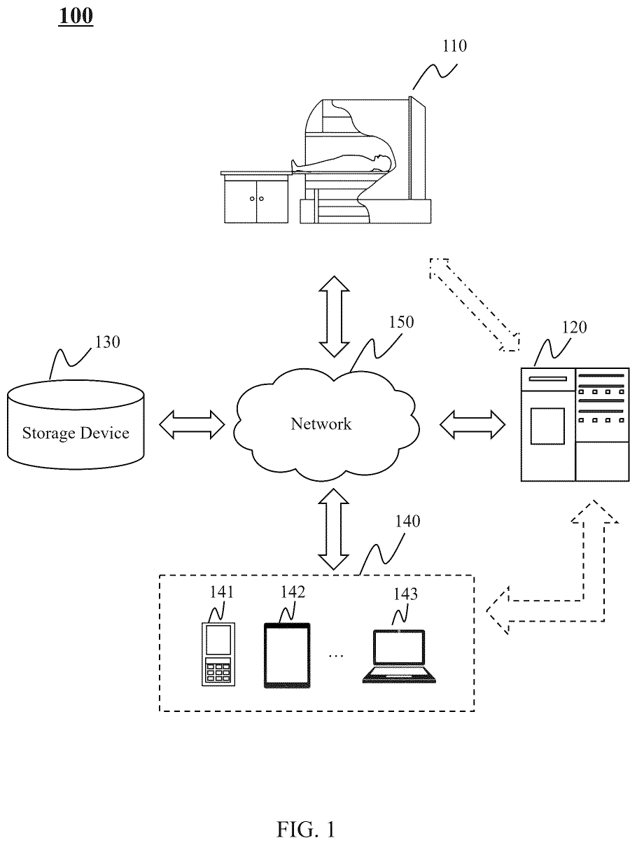

FIG. 1 is a schematic diagram illustrating an exemplary image processing system 100 according to some embodiments of the present disclosure. As shown in FIG. 1, the image processing system 100 may include an imaging device 110, a processing device 120, a storage device 130, one or more terminals 140, and a network 150. In some embodiments, the imaging device 110, the processing device 120, the storage device 130, and/or the terminal(s) 140 may be connected to and/or communicate with each other via a wireless connection, a wired connection, or a combination thereof.

The imaging device 110 may be configured to scan a target subject (or a part of the subject) to acquire medical image data associated with the target subject. The medial image data relating to the target subject may be used for generating a medical image (e.g., a PET image, etc.) of the target subject. The medical image may illustrate an internal structure and the health condition of the target subject. In some embodiments, the imaging device 110 may include a single-modality scanner and/or multi-modality scanner. The single modality scanner may include, for example, a positron emission tomography (PET) scanner. The multi-modality scanner may include, for example, a positron emission tomography-X-ray imaging (PET-X-ray) scanner, a positron emission tomography-computed tomography (PET-CT) scanner, a single-photon emission computed tomography-magnetic resonance imaging (SPECT-MRI) scanner, etc. It should be noted that the imaging device 110 described below is merely provided for illustration purposes, and is not intended to limit the scope of the present disclosure.

In some embodiments, the processing device 120 may be a single server or a server group. The server group may be centralized or distributed. The processing device 120 may process data and/or information obtained from the imaging device 110, the storage device 130, and/or the terminal(s) 140. For example, the processing device 120 may obtain PET data of the target object within a FOV of a PET imaging device and CT data of the target object within a FOV of a CT imaging device. The processing device 120 may obtain an attenuation image based on a target registration parameter and the CT data. The target registration parameter may characterize a mapping relationship registering the CT coordinate system and the PET coordinate system. The target registration parameter may be obtained based on background coincidence event data from the PET imaging device. The processing device 120 may also reconstruct a target PET image based on the PET data and the attenuation image. As another example, the processing device 120 may obtain PET data of the target object within a FOV of a positron emission computed tomography (PET) imaging device. The processing device 120 may obtain corrected PET data by correcting the PET data based on a target normalization correction factor. The target normalization correction factor is a normalization correction factor corresponding to a current state of the PET imaging device. The processing device 120 may determine the target normalization correction factor by the following operations. First background coincidence event data of the detector crystal of the PET imaging device is acquired at a predetermined time point. A first background energy response is obtained based on the first background coincidence event data. A variation of the first background energy response is generated based on the first background energy response and a first reference background energy response, and the first reference background energy response is obtained under a first reference system state of the PET imaging device. Then, in response to determining that the variation of the first background energy response is not greater than a variation threshold, a first reference normalization correction factor is designated as the target normalization correction factor, and the first reference normalization correction factor is obtained based on a phantom under the first reference system state of the PET imaging device. The processing device 120 may also reconstruct a target PET image based on the corrected PET data.

obtaining corrected PET data by correcting the PET data based on a target normalization correction factor, wherein the target normalization correction factor is a normalization correction factor corresponding to a current state of the PET imaging device

In some embodiments, the processing device 120 may be local or remote from the image processing system 100. In some embodiments, the processing device 120 may be implemented on a cloud platform. In some embodiments, the processing device 120 or a portion of the processing device 120 may be integrated into the imaging device 110 and/or the terminal(s) 140. It should be noted that the processing device 120 in the present disclosure may include one or multiple processors. Thus, operations and/or method steps that are performed by one processor may also be jointly or separately performed by the multiple processors.

The storage device 130 may store data, instructions, and/or any other information. In some embodiments, the storage device 130 may store data obtained from the imaging device 110, the processing device 120, and/or the terminal(s) 140. In some embodiments, the storage device 130 may store data and/or instructions that the processing device 120 may execute or use to perform exemplary methods described in the present disclosure. In some embodiments, the storage device 130 may include a mass storage device, a removable storage device, a volatile read-and-write memory, a read-only memory (ROM), or the like, or a combination thereof. In some embodiments, the storage device 130 may be implemented on a cloud platform. In some embodiments, the storage device 130 may be part of the imaging device 110, the processing device 120, and/or the terminal(s) 140.

The terminal(s) 140 may be configured to enable a user interaction between a user and the image processing system 100. In some embodiments, the terminal(s) 140 may be connected to and/or communicate with the imaging device 110, the processing device 120, and/or the storage device 130. In some embodiments, the terminal(s) 140 may include a mobile device 141, a tablet computer 142, a laptop computer 143, or the like, or a combination thereof. In some embodiments, the terminal(s) 140 may be part of the processing device 120 and/or the imaging device 110.

The network 150 may include any suitable network that can facilitate the exchange of information and/or data for the image processing system 100. In some embodiments, one or more components of the image processing system 100 (e.g., the imaging device 110, the processing device 120, the storage device 130, the terminal(s) 140, etc.) may communicate information and/or data with one or more other components of the image processing system 100 via the network 150.

It should be noted that the above description is intended to be illustrative, and not to limit the scope of the present disclosure. Many alternatives, modifications, and variations will be apparent to those skilled in the art. The features, structures, methods, and characteristics of the exemplary embodiments described herein may be combined in various ways to obtain additional and/or alternative exemplary embodiments. In some embodiments, the image processing system 100 may include one or more additional components and/or one or more components described above may be omitted. Additionally or alternatively, two or more components of the image processing system 100 may be integrated into a single component. For example, the processing device 120 may be integrated into the imaging device 110. As another example, a component of the image processing system 100 may be replaced by another component that can implement the functions of the component. However, those variations and modifications do not depart from the scope of the present disclosure.

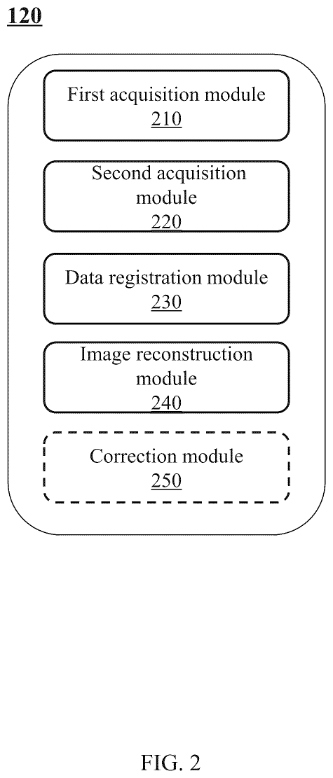

FIG. 2 is a block diagram illustrating an exemplary processing device 120 according to some embodiments of the present disclosure. As shown in FIG. 2, the processing device 120 includes a first acquisition module 210, a second acquisition module 220, a data registration module 230, and an image reconstruction module 240.

The first acquisition module 210 may be configured to acquire PET data of a target object within a FOV of a PET imaging device in a PET-CT system. More descriptions regarding obtaining the PET data may be found elsewhere in the present disclosure (e.g., operations 310 and the descriptions thereof).

The second acquisition module 220 may be configured to acquire CT data of the target object within a FOV of a CT imaging device in the PET-CT system. More descriptions regarding obtaining the CT data may be found elsewhere in the present disclosure (e.g., operations 320 and the descriptions thereof).

The data registration module 230 may be configured to generate an attenuation image based on a target registration parameter and the CT data. The target registration parameter characterizes a mapping relationship registering the CT coordinate system and the PET coordinate system. The target registration parameter is obtained based on background coincidence event data from the PET imaging device. More descriptions regarding generating the attenuation image may be found elsewhere in the present disclosure (e.g., operations 330 and the descriptions thereof).

The image reconstruction module 240 may be configured to reconstruct a target PET image based on the PET data and the attenuation image. More descriptions regarding reconstructing the target PET image may be found elsewhere in the present disclosure (e.g., operations 340 and the descriptions thereof).

In some embodiments, the processing device 120 further includes a correction module 250. The correction module 250 may be configured to obtain corrected PET data by correcting the PET data based on a target normalization correction factor. The target normalization correction factor refers to a normalization correction factor corresponding to a current state of the PET imaging device. The correction module 250 may determine the target normalization correction factor by the following operations. First background coincidence event data of a detector crystal of the PET imaging device is obtained at a predetermined time point. Based on the first background coincidence event data, a first background energy response is obtained. A variation of a first background energy response is generated based on the first background energy response and a first reference background energy response, and the first reference background energy response is obtained under a first reference system state of the PET imaging device. Then, in response to determining that the variation of the first background energy response is not greater than a variation threshold, a first reference normalization correction factor is designated as the target normalization correction factor, and the first reference normalization correction factor is obtained based on a phantom under the first reference system state of the PET imaging device. The image reconstruction module 240 may be configured to reconstruct the target PET image based on the corrected PET data. In response to determining that the variation of the first background energy response is greater than the variation threshold, a variation of a first normalization correction factor is generated based on the variation of the first background energy response and a predetermined relationship. The target normalization correction factor is determined based on the variation of the first normalization correction factor and the first reference normalization correction factor.

It should be noted that the above descriptions of the processing device 120 are provided for the purposes of illustration, and are not intended to limit the scope of the present disclosure. For persons having ordinary skills in the art, various modifications and changes in the forms and details of the application of the above system may occur without departing from the principles of the present disclosure. In some embodiments, the processing device 120 may include one or more other modules and/or one or more modules described above may be omitted. For example, the processing device 120 may not include the correction module 250. Additionally or alternatively, two or more modules may be integrated into a single module and/or a module may be divided into two or more units. For example, the first acquisition module 210 and the second acquisition module 220 may be integrated into a single module for obtaining the PET data and the CT data of the target object. However, those variations and modifications also fall within the scope of the present disclosure.

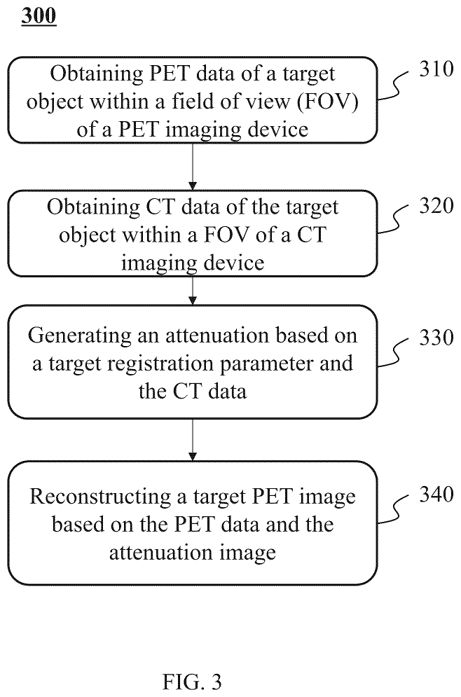

FIG. 3 is a flowchart illustrating an exemplary process for processing a medical image according to some embodiments of the present disclosure. In some embodiments, process 300 may be executed by the image processing system 100 and/or the processing device 120. For example, the process 300 may be implemented as a set of instructions (e.g., an application) stored in a storage device (e.g., the storage device 130 illustrated in FIG. 1). In some embodiments, the processing device 120 of the image processing system 100 and/or one or more modules of the processing device 120 may execute the set of instructions and may accordingly be directed to perform the process 300. In some embodiments, the process 300 may be implemented in a PET-CT system (e.g., the imaging device 110) including a CT imaging device and a PET imaging device.

In 310, PET data of a target object within a FOV of the PET imaging device is obtained. In some embodiments, operation 310 may be performed by a first acquisition module 210 shown in FIG. 2.

The target object (also referred to as a target scanning object) is a scanning object of the PET-CT system. For example, the target object may include an organism, a phantom, or the like. The organism may be a human body, a small animal, or the like. The phantom may be of various materials and shapes. For example, the phantom may include a water phantom, a gel material phantom, a wood phantom, a cylinder, a rectangle, or the like. Merely by way of example, the target object may be a human body and/or a water phantom.

The PET data may include at least one of PET scanned data or a PET image. In some embodiments, the processing device 120 may scan the target object within the FOV of the PET imaging device to obtain the PET scanned data, and designate the PET scanned data as the PET data of the target object. The PET scanned data includes coincidence event data. The coincidence event data refers to data of a coincidence event corresponding to a particular energy level, e.g., a coincidence event corresponding to an energy level of 511 keV, a coincidence event corresponding to an energy level of 662 keV, or the like. In some embodiments, the processing device 120 may perform an image reconstruction based on the PET scanned data, and designate the reconstructed PET image as the PET data of the target object.

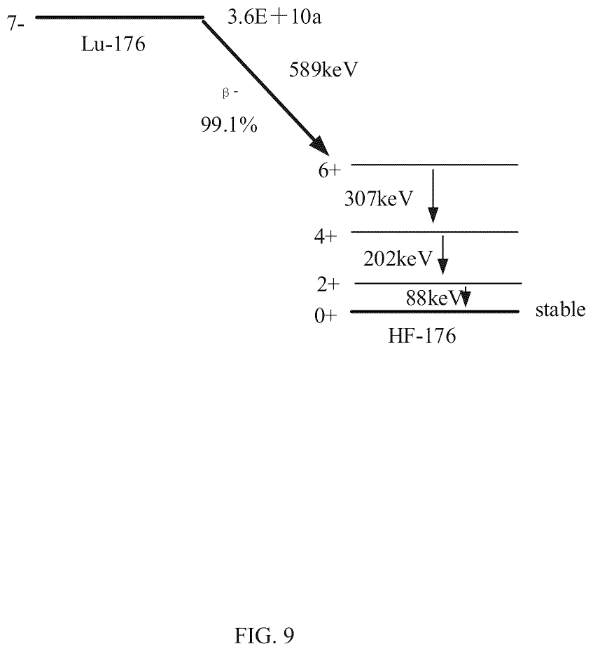

In some embodiments, the processing device 120 may obtain background radiation data of the PET imaging device by performing scanning without an object or scanning a phantom, etc. The background radiation data includes background coincidence event data. A background coincidence event (also referred to as a background event) refers to a coincidence event that is generated by spontaneous background radiation from a crystal in the PET imaging device, i.e., a radiation signal received by the coincidence event is generated by background radiation. Common PET systems use a lutetium orthosilicate (LSO) crystal or a lutetium yttrium orthosilicate (LYSO) crystal (i.e., scintillator crystals) as a detector crystal. The LSO crystal or the LYSO crystal contains the isotope Lu-176, which is capable of generating spontaneous background radiation. FIG. 9 is a schematic diagram illustrating a decay energy level of exemplary Lu-176 according to some embodiments of the present disclosure. As shown in FIG. 9, the decay of Lu-176 consists of simultaneous beta (β) decay and cascade gamma (γ) decay, and the maximum energy of β decay is 589 keV, and the energies of γ decay are 307 keV, 202 keV, and 88 keV, which may be utilized to screen the background event by utilizing the simultaneity of β decay and γ decay and energies corresponding to β decay and γ decay. The present disclosure does not limit the type of the detector crystal of the PET imaging device. Merely by way of example, the detector crystal of the PET imaging device may be employed including, but not limited to, sodium iodide (NaI), bismuth germanate (BGO), lutetium orthosilicate (LSO), and lutetium yttrium orthosilicate (LYSO), etc.

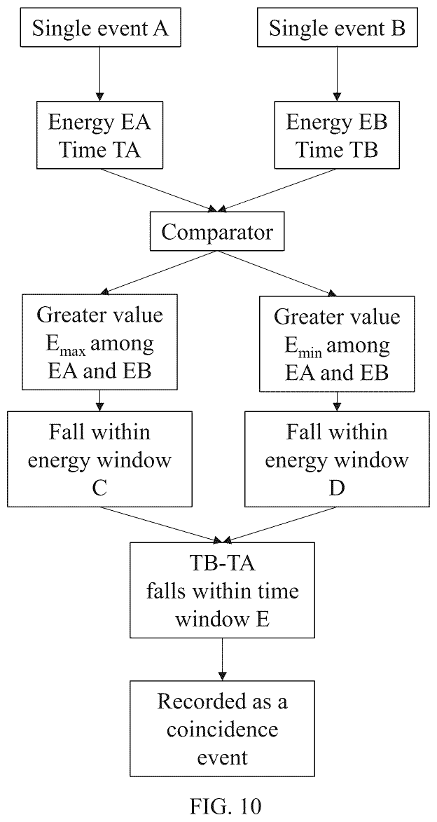

In some embodiments, the processing device 120 may obtain a background radiation signal of the PET imaging device to obtain the background coincidence event data. The processing device 120 may obtain the background coincidence event based on arrival energies and arrival times of β rays and γ rays in the background coincidence event data. The manner of determining the background coincidence event is simple, easy to operate, and highly accurate. FIG. 10 is a schematic diagram illustrating a discrimination process of an exemplary background coincidence event in a PET imaging device according to some embodiments in the present disclosure. As shown in FIG. 10, at a certain point in time, a detector (e.g., detector A) receives a single event A and another detector (e.g., detector B) receives a single event B. An energy and an arrival time of the single event A are EA and TA, respectively, and an energy and an arrival time of the single event B are EB and TB, respectively, and TA is larger than TB (event B occurs before event A). The processing device 120 may select, via a comparator, the greater one of the energy EA and the energy EB as a greater value Eₘₐₓ, and the smaller one of the energy EA and the energy EB as a smaller value Eₘᵢₙ. If the larger value Eₘₐₓ (e.g., the energy EA) falls within an energy window C (i.e., a coincidence window), the smaller value Eₘᵢₙ (e.g., the energy EB) falls within an energy window D, and an arrival time TA-TB falls within a time window E, the event is recorded as a background coincidence event. The energy windows are energy ranges, and the energy windows may be used to determine if a specific event (e.g., the coincidence event, etc.) has occurred. Exemplarily, the value of the energy window E may be taken as shown in equation (1):

$$[ max ( {E}_{β} , {E}_{γ} ) - 3 σ , max ( {E}_{β} , {E}_{γ} ) + 3 σ ] , \qquad ( 1 )$$

the value of the energy window D is shown in equation (2):

$$[ min ( {E}_{β} , {E}_{γ} ) - 3 σ , min ( {E}_{β} , {E}_{γ} ) + 3 σ ] . \qquad ( 2 )$$

The time window is a time range. The value of the time window E is shown in equation (3):

$$[ - Δ {T}_{max} - 3 {σ}^{′} , Δ {T}_{max} + 3 {σ}^{′} ] . \qquad ( 3 )$$

In equation (1) and equation (2), E_{β} denotes a deposition energy of β rays within the crystal, E_{γ} denotes a deposition energy of γ rays within the crystal, and a denotes a standard deviation of Gaussian energy distribution of the system. In equation (3), ΔTₘₐₓ denotes a maximum possible value of the absolute value of a difference between the arrival time of β rays and the arrival time of γ rays at the detector, and σ′ denotes the standard deviation of the Gaussian time distribution of the system.

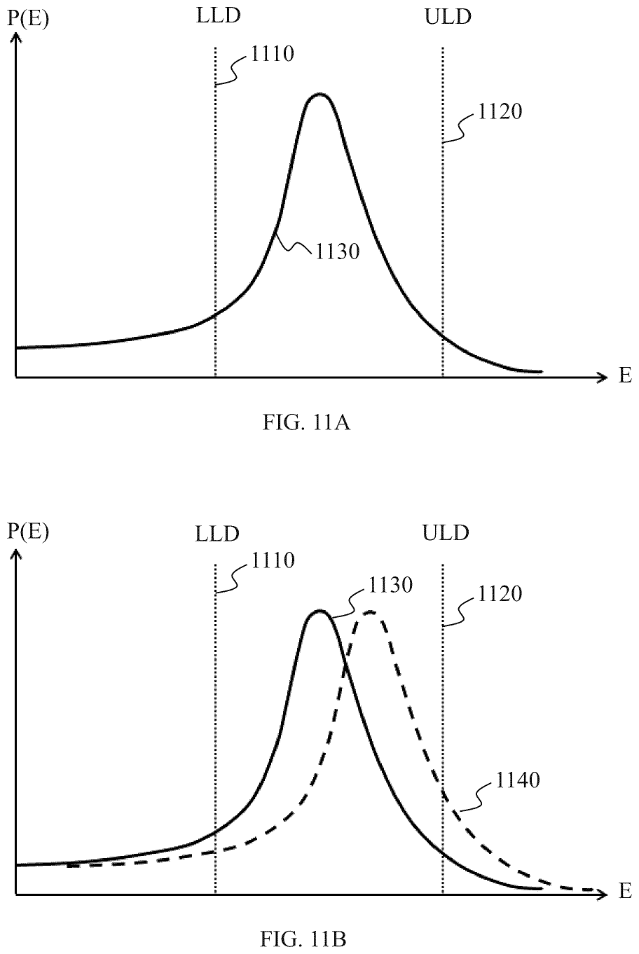

In some embodiments, the background radiation energy (e.g., the background γ decay energy) may be 307 keV, 202 keV, or 88 keV, etc. The background radiation energy is also referred to as the background energy. A background energy response is an energy response of the background radiation. In some embodiments, the energy response may be expressed as a function, referred to as an energy response function. For example, the energy response function may be functions shown in a curve 1130, a curve 1140 in FIG. 11A and FIG. 11B.

Taking a certain radiation source (e.g., a radiation source capable of producing a positron annihilation event or a pair of β-γ background events) irradiating a response line or a crystal as an example, the energy distribution of all incident single events (i.e., a single coincidence event) is recorded, and the energy response of the response line or the crystal to the radiation source may be obtained. For background coincidence data, time-of-flight information may be utilized to pick out β decay single events or γ decay single events. FIG. 11A is a schematic diagram illustrating an exemplary background energy response according to some embodiments of the present disclosure. In FIG. 11A, the horizontal coordinate represents the energy, the vertical coordinate represents a probability of occurrence of a single event at a certain energy (e.g., 307 keV, 511 keV), the dashed line 1110 represents a lower threshold of an energy window (LLD), the dashed line 1120 represents an upper threshold of an energy window (ULD), and the solid curve 1130 represents an energy response function P(E).

In 320, CT data of the target object within a FOV of the CT imaging device is obtained. In some embodiments, operation 320 may be performed by the second acquisition module 220 shown in FIG. 2.

The CT data may include at least one of CT scanned data and a CT image. In some embodiments, the processing device 120 may scan the target object within the FOV of the CT imaging device to obtain the CT scanned data, and designate the CT scanned data as the CT data of the target object. In some embodiments, the processing device 120 may perform the image reconstruction based on the CT scanned data, and designate the reconstructed CT image as the CT data of the target object.

In 330, an attenuation image is generated based on the target registration parameter and the CT data. In some embodiments, operation 330 may be performed by the data registration module 230 shown in FIG. 2.

The registration operation refers to an operation of converting image data in different reference systems (coordinate systems) to the same reference system. The registration parameter refers to a parameter used to register different medical image data. For example, the registration parameter may be a parameter used to register image data obtained from two scans of the same type of medical imaging device (e.g., the imaging device for the two scans is the CT imaging device). As another example, the registration parameter may be a parameter used to register image data from different types of medical imaging devices. The registration parameter may include a rotation matrix and a translation vector, etc. The rotation matrix refers to a parameter used to convert coordinates of image pixel points from one coordinate system to another coordinate system by rotation, represented as a matrix. The translation vector refers to a parameter used to convert the coordinates of the image pixel points from one coordinate system to another coordinate system by translation, represented as a vector.

The target registration parameter characterizes a mapping relationship registering the CT coordinate system and the PET coordinate system. For example, the target registration parameter may be a parameter used to register the CT image acquired at operation 320 to the PET image acquired at operation 310.

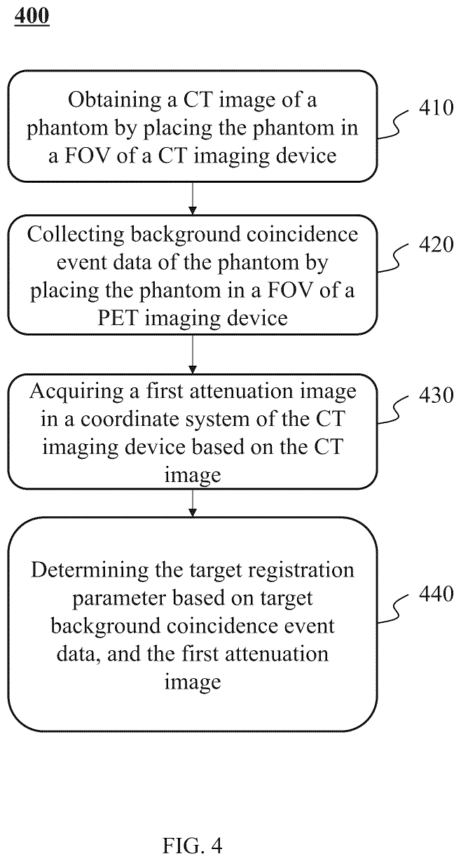

In some embodiments, the processing device 120 may obtain the target registration parameter in various ways. For example, the processing device 120 may obtain the target registration parameter via a predetermined algorithm, a machine learning model, and manual registration. In some embodiments, the processing device 120 may obtain the target registration parameter based on background radiation data of the PET device. Detailed descriptions regarding obtaining the target registration parameter based on the background radiation data of the PET device may be found in FIG. 4 and related descriptions thereof.

In some embodiments, the processing device 120 may register the CT data and the PET data based on the target registration parameter, i.e., converting the CT image acquired at operation 320 and the PET image acquired at operation 310 to the same coordinate system. For example, the processing device 120 may convert the CT image in a coordinate system of the CT imaging device (a coordinate system established with the CT imaging device as a reference) to a coordinate system of the PET imaging device (a coordinate system established with the PET imaging device as a reference) based on the target registration parameter, thereby registering the CT data to the PET data. After the registration is completed, the processing device 120 may obtain the attenuation image based on the registered CT image. The processing device 120 may use any feasible means to obtain the attenuation image based on the CT image, which is not limited by present disclosure. For example, the processing device 120 may obtain the attenuation image through a machine learning model or using a predetermined algorithm. For example, the predetermined algorithm may include the Fourier transform, backprojection method, etc.

In 340, a target PET image is reconstructed based on the PET data and the attenuation image. In some embodiments, operation 340 may be performed by the image reconstruction module 240 shown in FIG. 2.

In some embodiments, the processing device 120 may reconstruct a PET image based on the PET scanned data and the attenuation image, and designate the generated PET reconstructed image as the target PET image.

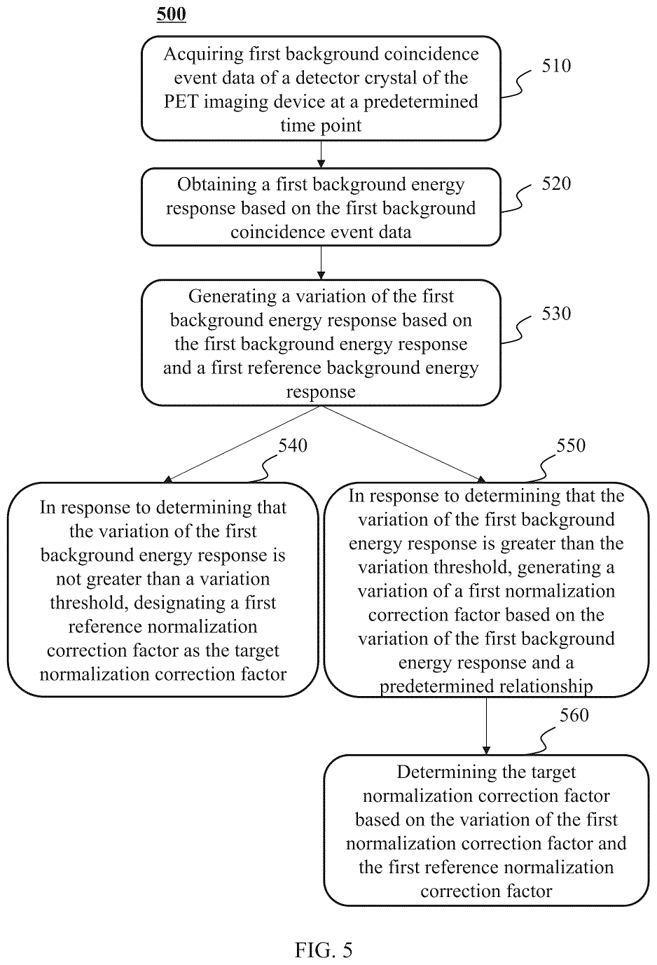

In some embodiments, the processing device 120 may correct the PET data and then reconstruct the PET image based on the corrected PET data and the attenuation image to generate the target PET image. For example, the processing device 120 may obtain the corrected PET data by correcting PET scanned data based on a target normalization correction factor. The target normalization correction factor refers to a normalization correction factor corresponding to a current system state of the PET imaging device. The target normalization correction factor is obtained based on the background radiation data of the PET imaging device. Specifically, the processing device 120 may obtain background energy responses (e.g., a first background energy response and a first reference background energy response) of the detector crystal under the current system state and a reference system state (e.g., a first reference system state) of the PET imaging device, respectively. The processing device 120 may generate a variation of the background energy response (e.g., a variation of the first background energy response) based on the two background energy responses. The processing device 120 may determine whether the variation of background energy response is greater than a variation threshold. Further, in response to determining that the variation of the background energy response is not greater than the variation threshold, the processing device 120 may designate the normalization correction factor (e.g., the first reference normalization correction factor) under the reference system state of the PET imaging device as the target normalization correction factor. In response to determining that the variation of the first background energy response is greater than the variation threshold, the processing device 120 may generate a variation of a first normalization correction factor based on the variation of the first background energy response and a predetermined relationship and determine the target normalization correction factor based on the variation of the first normalization correction factor and the first reference normalization correction factor. Descriptions regarding determining the target normalization correction factor may be found in FIG. 5 and related descriptions thereof.

The medical image processing method provided in the present disclosure eliminates the need to perfuse a phantom or prepare a solid registration source, simplifies the operation process, saves manpower and material resources, improves the efficiency of registration, and establishes a solid foundation for rapid registration of the PET image and the CT image. The method of the present disclosure may reduce the radiation damage of the radiation source to the operator. Because there is no need to perfuse the phantom or prepare the solid registration source, the method of the present disclosure may significantly reduce requirements for the hardware of the PET-CT imaging device and may be suitable for the PET-CT imaging device without an external source mounting device and motion device. In the method of the present disclosure, the attenuation correction is performed without increasing any hardware cost, has good applicability and is easy to deploy and implement.

FIG. 4 is a flowchart illustrating an exemplary process for obtaining a target registration parameter according to some embodiments shown in the present disclosure. In some embodiments, at least part of the process 400 may be performed to achieve at least part of operation 330 as described in connection with FIG. 3. For example, the processing device 120 or the data registration module 230 may obtain the target registration parameter by performing at least part of the process 400.

In 410, a CT image of a phantom is obtained by placing the phantom in a FOV of a CT imaging device.

In some embodiments, the processing device 120 may place the phantom within the FOV of the CT imaging device (e.g., a CT imaging device in a PET-CT system), scan the phantom, and obtain scan data of the phantom. The processing device 120 may perform an image reconstruction based on the scan data to obtain a reconstructed image, and designate the reconstructed image as the CT image of the phantom.

In some embodiments, the phantom may be a phantom that includes or does not include a radiation source. The present disclosure does not limit the specific shape of the phantom. For example, the phantom may be uniformly cylindrical, circular, spherical, etc.

In 420, background coincidence event data of the phantom is collected by placing the phantom in the FOV of the PET imaging device.

In some embodiments, the processing device 120 may place the phantom in the FOV of the PET imaging device (e.g., a PET imaging device in a PET-CT system), scan the phantom, and collect the background coincidence event data from the PET imaging device. More descriptions regarding the background coincidence event data may be found in the related description of operation 310.

In some embodiments, a collection amount of the background coincidence event data may be determined based on a current remaining computing resource of the PET-CT system. For example, the collection amount of the background coincidence event data is positively correlated (e.g., proportional) to the current remaining computing resource of the PET-CT system. The remaining computing resource may be determined by a utilization rate of each central processing unit (CPU) of the PET-CT system. The remaining computing resource is negatively correlated to the utilization rate of each CPU of the PET-CT system. For example, the current remaining computing resource of the system is equal to a sum of a remaining utilization rate of each CPU, i.e., the remaining utilization rate=1−the utilization rate of the CPU. Merely by way of example, the higher the current utilization rate of the CPU, the fewer the remaining computing resource, and the smaller the collection amount of the background coincidence event data. The lower the current utilization rate of the CPU, the more the remaining computing resource, and the larger the collection amount of the background coincidence event data.

In 430, a first attenuation image in a coordinate system of the CT imaging device is acquired based on the CT image.

In some embodiments, the processing device 120 may obtain an attenuation image in the coordinate system of the CT imaging device based on the CT image, and designate the obtained attenuation image as the first attenuation image. The processing device 120 may obtain the attenuation image based on the CT image in any feasible way, which is not limited in the present disclosure. For example, the processing device 120 may obtain the attenuation image through a machine learning model or using a predetermined algorithm.

In 440, the target registration parameter is determined based on target background coincidence event data, and the first attenuation image.

In some embodiments, the processing device 120 may obtain the target background coincidence event data based on the background coincidence event data. For example, the processing device 120 may designate the background coincidence event data as the target background coincidence event data. As another example, the processing device 120 may obtain background coincidence event data after noise reduction by performing noise reduction on the background coincidence event data. Further, the processing device 120 may designate the background coincidence event data after noise reduction as the target background coincidence event data. By designating the background coincidence event data after noise reduction as the target background coincidence event data, noise impact may be reduced and data accuracy of the background coincidence event data may be improved, thereby improving the accuracy of the registration.

In some embodiments, the processing device 120 may process the background coincidence event data by noise reduction in various ways. For example, the processing device 120 may perform noise reduction through filtering. As another example, the processing device 120 may input the background coincidence event data into a trained neural network model for noise reduction.

In some embodiments, the processing device 120 may perform noise reduction on the background coincidence event data by merging an original response line. Specifically, the background coincidence event data of the phantom may include a plurality of original response lines. Each original response line may be formed by connecting two crystals, and the connection is a virtual connection. The processing device 120 may merge at least two target response lines in these original response lines into one combined response line. The processing device 120 may obtain the background coincidence event data after noise reduction based on the background coincidence event data received by at least one combined response line. A single original response line has a weak strength of a background radiation signal, resulting in low accuracy of a count of background events. In some embodiments of the present disclosure, by merging at least two target response lines in the original response lines into the combined response line, the background coincidence event data after noise reduction is obtained based on the background coincidence event data received by the combined response line, increasing the count of background events received on a single response line, effectively reducing statistical noise, and thereby improving the accuracy of the obtained registration parameter.

In some embodiments, the target response line may be determined in the following manner. For any one of the original response lines in the background coincidence event data of the phantom, in response to determining that a proportion of background coincidence event data that is received by the original response line during a preset time period (e.g., 30 minutes, 60 minutes, etc.) and satisfies a preset condition is greater than a preset proportion threshold (e.g., 80%, 90%, etc.), the processing device 120 may designate the original response line as the target response line. The preset condition may be that the energy of the β rays and/or the γ rays in the background coincidence event data received by the original response line is lower than a preset threshold. The β rays and γ rays may correspond to different preset thresholds, respectively. The preset threshold may be determined based on the energy levels of the β rays and the γ rays. For example, the preset threshold corresponding to β rays may be 589 keV and the preset threshold corresponding to γ rays may be 88 keV. By counting the proportion of β rays and/or γ rays whose energy is lower than a set value (e.g., the preset threshold) in the background coincidence event data received by the original response line over a time period, an original response line with a weaker strength of the background radiation signal may be accurately determined.

In some embodiments, for each of the at least two target response lines, the processing device 120 may merge the target response line and any one other original response lines having a distance within a preset range (e.g., 10 cm, 20 cm, etc.) from the target response line into one combined response line. The other original response lines are non-target response lines among the original response lines of the background coincidence event data of the phantom. Merely by way of example, the original response line A and the original response line B are both connected by two crystals, the original response line A corresponds to crystal 1 and crystal 2, the original response line B corresponds to crystal 3 and crystal 4, and a distance between the original response line A and the original response line B is an average of distances between crystals in the original response line A and crystals in the original response line B, which may be expressed as (L13+L14+L23+L24)/4, where L13 denotes a distance between crystal 1 and crystal 3, and L14 denotes a distance between crystal 1 and crystal 4, L23 denotes a distance between crystal 2 and crystal 3, and L24 denotes a distance between crystal 2 and crystal 4. The strength of the background radiation signal on the combined response line may be significantly increased by merging the target response line and the non-target response line that is closer to the target response line.

In some embodiments, the processing device 120 may perform noise reduction on the background coincidence event data by smoothing or using AI noise reduction algorithm for processing the background coincidence event data. For example, the processing device 120 may obtain the background coincidence event data after noise reduction by smoothing or using AI noise reduction algorithm for processing original background coincidence event data received by original response lines in the background coincidence event data of the phantom. By smoothing or using AI noise reduction algorithm for processing the original background coincidence event data, the difficulty of processing the background coincidence event data may be reduced and the processing efficiency may be improved.

In some embodiments, the processing device 120 may iteratively update, based on the target background coincidence event data, the first attenuation image, and a predetermined registration parameter, the predetermined registration parameter using a maximum likelihood estimation (MLE) function until a preset end condition is satisfied. The preset end condition may include a count of iterations reaching a preset value, a value of the maximum likelihood estimation function starting to decline, or the like, which is not limited to the present disclosure. The processing device 120 may designate the predetermined registration parameter when the value of the MLE function is maximized during the iteration process, as the target registration parameter. By using the MLE function to obtain the target registration parameter by iterative updating, the target registration parameter may be determined quickly based on the predetermined registration parameter, improving the efficiency of determining the target registration parameter and improving the accuracy of the registration parameter, thus improving the registration efficiency and registration accuracy.

In some embodiments, the target background coincidence event data may include not only the background coincidence event data of the phantom, but also background coincidence event data obtained by scanning without an object. Then the processing device 120 may obtain a second attenuation image based on the target background coincidence event data, and obtain the predetermined registration parameter based on the first attenuation image and the second attenuation image by using a registration algorithm. Specifically, the processing device 120 may perform an image reconstruction based on the target background coincidence event data, and then obtain the second attenuation image based on the reconstructed image. In some embodiments, the processing device 120 may directly use the predetermined registration parameter as the target registration parameter.

In some embodiments, during the iteration described above, the processing device 120 may obtain the predetermined registration parameter using an optimization algorithm. The processing device 120 may update the estimated values of an predetermined registration parameter R and an predetermined registration parameter T by the optimization algorithm. The optimization algorithm may be, for example, a gradient descent algorithm, a Newton algorithm, a conjugate gradient algorithm, etc., which is not limited in the present disclosure.

Merely by way of example, the MLE function may be shown in equation (4) as follows:

$$L ( R , T ) = {\sum }_{i} [ {y}_{i} log ( {\overline{y}}_{i} ) - {\overline{y}}_{i} ] , \qquad ( 4 )$$

where R and T denote the registration parameters, R denotes a rotation matrix with 3 rows and 3 columns, and T denotes a translation vector with 3 rows and 1 column, L denotes the MLE function, yᵢ denotes a measured count rate of background events on the ith response line, and y̅ᵢ denotes an expected count rate of background events.

In some embodiments, when the target background coincidence event data includes not only the background coincidence event data of the phantom, but also background coincidence event data obtained by scanning without an object, and then the expected count rate y̅ᵢ of background events in equation (4) may be obtained from equation (5) as follows:

$${\overline{y}}_{i} = {a}_{i} {b}_{i} + {s}_{i} , \qquad ( 5 )$$

where aᵢ denotes an attenuation effect factor on the ith response line, sᵢ denotes a estimated value of background scattering events on the ith response line, and bᵢ denotes a measured count rate of background events on the ith response line during the scanning without an object.

The attenuation effect factor aᵢ in equation (5) may be obtained from equation (6) as follows:

$${a}_{i} = exp ( - {\sum }_{j = 1}^{J} {l}_{ij} {μ}_{j} ) , \qquad ( 6 )$$

where lᵢⱼ denotes an intersection length of the ith response line passing through the jth voxel, J denotes an number of the response voxels, and μⱼ denotes an attenuation coefficient value of the jth voxel in the attenuation image in the coordinate system of the PET imaging device. For example, μⱼ can be the second attenuation image, the second attenuation image is an attenuation image in the coordinate system of the PET imaging device.

A relationship between the first attenuation image and the second attenuation image may be shown in equation (7) as follows:

$$μ = R {μ}_{CT} + T , \qquad ( 7 )$$

where μ denotes the second attenuation image in the coordinate system of the PET imaging device, μ_{CT} denotes the first attenuation image in the coordinate system of the CT imaging device, the second attenuation image (μ) is obtained by transforming the first attenuation image (μ_{CT}) into the coordinate system of the PET imaging device, and the meanings of R and T are the same as those in equation (4). By maximizing the MLE function L in equation (4), the estimated value of the rotation matrix R and the translation vector T may be obtained as shown in equation (8) as follows:

$$( \hat{R} , \hat{T} ) = arg max ( L ) , \qquad ( 8 )$$

where \hat{R} denotes an estimated value of the rotation matrix R, and \hat{T} denotes an estimated value of the translation vector T. As previously described, the processing device 120 may obtain estimated values of the rotation matrix R and the translation vector T using the optimization algorithm(e.g., equation (8)). The optimization algorithm may be, for example, a gradient descent algorithm, a Newtonian algorithm, a conjugate gradient algorithm, etc. The processing device 120 may obtain a plurality of maximum values of the MLE function L through a plurality of rounds of iterations, and determine the rotation matrix \hat{R} and the translation vector \hat{T} corresponding to each maximum value through equation (8). At the end of the iteration, the processing device 120 may designate the rotation matrix \hat{R} and the translation vector \hat{T} corresponding to the largest one of these maximum values of the MLE L as the target registration parameter.

In some embodiments, the processing device 120 may divide the target background coincidence event data into a plurality of groups of sub-background coincidence event data based on an acquisition time period, and obtain the target registration parameter based on the plurality of groups of sub-background coincidence event data.

Specifically, for each group of sub-background coincidence event data, the processing device 120 may iteratively update, based on the group of sub-background coincidence event data, the first attenuation image, and the predetermined registration parameter using the MLE function until the preset end condition is satisfied. The processing device 120 may designate the predetermined registration parameter when the value of the MLE function is maximized during the iteration, as a reference registration parameter for the group of sub-background coincidence event data. The reference registration parameter of the plurality of groups of sub-background coincidence event data is weighted and summed to obtain the target registration parameter. For example, there are three groups of sub-background coincidence events A, B, and C. Reference registration parameters corresponding to the three groups A, B, and C are [a11, a12, a13; a21, a22, a23; a31, a32, a33], [b11, b12, b13; b21, b22, b23; b31, b32, b33], and [c11, c12, c13; c21, c22, c23; c31, c32, c33], with corresponding weights m1, m2, and m3, respectively. Then the target registration parameter after weighted summation is [(m1×a11+m2×b11+m3×c11), (m1×a12+m2×b12+m3×c12), (m1×a13+m2×b13+m3×c13), (m1×a21+m2×b21+m3×c21), (m1×a22+m2×b22+m3×c22), (m1×a23+m2×b23+m3×c23), (m1×a31+m2×b31+m3×c31), (m1×a32+m2×b32+m3×c32), (m1×a33+m2×b33+m3×c33)].

In some embodiments, for each group of sub-background coincidence event data, the processing device 120 may determine, based on an average arrival time difference between the β rays and the γ rays in the group of sub-background coincidence event data, a corresponding weight of the reference registration parameter for the group of sub-background coincidence event data. For example, the larger the average arrival time difference, the smaller the weight.

In some embodiments, for each group of sub-background coincidence event data, the processing device 120 may determine, based on an average energy of the β rays or an average energy of the γ rays in the background coincidence event data, the corresponding weight of the reference registration parameter for the group of sub-background coincidence event data. For example, the higher the average energy, the higher the weight.

In some embodiments, the processing device 120 may iteratively update, based on the plurality of groups of sub-background coincidence event data, the first attenuation image, and the predetermined registration parameter, the predetermined registration parameter using the MLE function until the preset end condition is satisfied. In each round of iteration, for each group of the plurality of groups of sub-background coincidence event data, the processing device 120 may determine, based on the group of sub-background coincidence event data, the first attenuation image, and the predetermined registration parameter, the value of the MLE function corresponding to the sub-background coincidence event data. The processing device 120 may weigh and sum the values of the MLE function corresponding to the plurality of groups of sub-background coincidence event data to obtain a target function value. The processing device 120 may designate the predetermined registration parameter when the target function value is maximized as the target registration parameter.

In some embodiments, for each group of sub-background coincidence event data, the processing device 120 may determine, based on the average arrival time difference of the β rays and the γ rays in the group of sub-background coincidence event data, a weight of the value of the MLE function corresponding to the group of sub-background coincidence event data. For example, the larger the average arrival time difference, the smaller the weight.

In some embodiments, for each group of sub-background coincidence event data, the processing device 120 may determine, based on the average energy of the β rays or the average energy of the γ rays in the background coincidence event data, a weight of the value of the MLE function corresponding to the group of sub-background coincidence event data. For example, the higher the average energy, the higher the weight.

In some embodiments, the processing device 120 may generate a plurality of candidate registration parameters, and each of the plurality of candidate registration parameters includes a parameter of the rotation matrix and a parameter of the translation vector. The manner in which the candidate registration parameters are generated is not limited to the present disclosure. The processing device 120 may perform a plurality of rounds of iterations on the generated plurality of candidate registration parameters. In each of at least one of the plurality of rounds of iterations, the processing device 120 may transform existing candidate registration parameters of a current round of iteration to obtain a first candidate registration parameter, and add the first candidate registration parameter to the existing candidate registration parameters. The transformation includes exchanging values of the same type of parameter in different existing candidate registration parameters. For each of the existing candidate registration parameters in the current round of iteration, the processing device 120 may predict, using an effect prediction model, a registration effect value of the candidate registration parameter based on the target background coincidence event data, the first attenuation image, the candidate registration parameter, and the PET data of the phantom. The effect prediction model is a machine learning model. The processing device 120 may designate a candidate registration parameter that has a registration effect value greater than the effect threshold as an existing candidate registration parameter for the next round of iteration. The processing device 120 may select a candidate registration parameter with the largest registration effect value as the target registration parameter from all candidate registration parameters in the plurality of rounds of iterations.

Merely by way of example, a registration effect value of a candidate registration parameter may be determined in the following manner. Under the candidate registration parameter, the processing device 120 may obtain a reference second attenuation image through the equation (7), determine a matching degree between each reference point in the reference second attenuation image and a corresponding reference point in actual PET data, and weight and sum matching degrees corresponding to all the reference points as the registration effect value of the candidate registration parameter. For example, the registration effect value of the candidate registration parameter=a₁×matching degree 1+a₂×matching degree 2+ . . . . +aₙ×matching degree n, where n denotes a count of reference points and aₙ-aₙ denote weights of the reference points. The reference points refer to pixel points included in both the first attenuation image, the reference second attenuation image, and the actual PET data. The matching degree between each reference point in the reference second attenuation image and the corresponding reference point in the actual PET data may be determined in various ways, e.g., may be determined manually, which is not limited in the present disclosure.

An operating environment may affect the accuracy and performance of the device and then affect the quality of the imaging, thereby affecting the difficulty of the registration. In some embodiments, in addition to the target background coincidence event data, the first attenuation image, the candidate registration parameters, and the PET data of the phantom, an input of the effect prediction model may include an operating environment of the PET imaging device, and an operating environment of the CT imaging device. The operating environment may include at least one of temperature, humidity, or excitation voltage.

The quality of imaging may be affected by excessive failures or repairs of the PET imaging device and/or the CT imaging device. To compensate for or minimize the degradation of the quality of the imaging due to the malfunctioning or repair of the PET imaging device and maintain the quality of the attenuation image obtained after the final registration, the registration effect may be ensured by increasing an effect threshold. In some embodiments, the effect threshold may be determined based on a count of malfunctions or repairs of the PET imaging device and/or the CT imaging device. For example, the effect threshold may be positively correlated with the count of malfunctions or repairs of the PET imaging device and/or the CT imaging device.

FIG. 5 is a flowchart illustrating an exemplary process for determining a target normalization correction factor according to some embodiments of the present disclosure. In some embodiments, at least part of the process 500 may be performed to achieve at least part of operation 340 as described in connection with FIG. 3. For example, the processing device 120 or the image reconstruction module 240 may determine the target normalization correction factor by performing at least part of the process 500.

A medical scanning system refers to a system that is capable of obtaining a medical image by scanning. For example, a PET system, a PET-CT system, or the like. When a system state of the medical scanning system changes, an energy response of the same response line or crystal to the same radiation source may change. In some embodiments, a change in the system state may include one or more of changes in the external environment, crystal aging, silicon photomultiplier (SiPM) gain, and light harvesting properties from the crystal to the SiPM. The normalization correction factor refers to information used in the medical scanning system for normalization correction, which may be a fixed value or a value that changes with the system state. The normalization correction of the medical scanning system is essentially the normalization correction of scan data (e.g., background coincidence event data, coincidence event data of a target object) of the medical scanning system using a normalization correction factor. In some embodiments, when the medical scanning system is a system that is a combination of a plurality of medical imaging devices, the change in the system state refers to the change in only some of the imaging devices. Taking the PET-CT system as an example, the change in the system state may be a change in a system state of a PET device in the PET-CT system.

In some embodiments, the normalization correction factor may include at least one of an axial profile correction factor, a detector ring pair detection efficiency correction factor, an annular profile correction factor, a crystal detection efficiency correction factor, a time-of-flight correction factor, or the like. With the plurality of normalization correction factors, the medical scanning system may be fully and accurately corrected.

FIG. 11B is a schematic diagram illustrating an exemplary change in energy response due to a change in a system state according to some embodiments of the present disclosure. FIG. 11B may represent the change in energy response following the change in the system state shown in FIG. 11A. As shown in FIG. 11B, the solid line 1130 represents an energy response function P(E) before the change in the system state occurs, and the dashed line 1140 represents an energy response function P′(E) of the same response line or crystal to the same radiation source after the change in the system state. It may be seen that after the change in the system state, an area under the energy response function between a lower threshold 1110 of an energy window and an upper threshold 1120 of the energy window may change, i.e., a count of coincidence events of the response line or crystal under irradiation from the radiation source of the same intensity may change, which indicates that the normalization correction factor of the response line or crystal may change. Generally, if the system state changes, a positron annihilation energy response and a background energy response may change similarly, i.e., the change in the background energy response is correlated with the change in the normalization correction factor. In some embodiments, the processing device 120 may obtain a relationship between a variation of the background energy response and a variation of the normalization correction factor under different states of the medical scanning system. The relationship includes a functional relationship, a mapping relationship, etc. The functional relationship can be expressed as a function. The mapping relationship can be expressed as a mapping table. The present disclosure will use the example of the functional relationship as a means of the relationship to illustrate.

In some embodiments, the change in the system state may be performed in various ways, such as by passive waiting or active control. For example, as time progresses, the crystal may undergo aging, leading to the change in the system state. A part of the crystal may be chosen to adjust a gain of a corresponding silicon photomultiplier (SiPM) to change the system state. As another example, as the external environment (e.g., temperature, humidity, etc.) changes, the system state may change, and the system state may be changed by actively or passively changing environmental parameters such as temperature and humidity. As yet another example, the system state may be changed by actively changing a parameter such as the excitation voltage of the system.

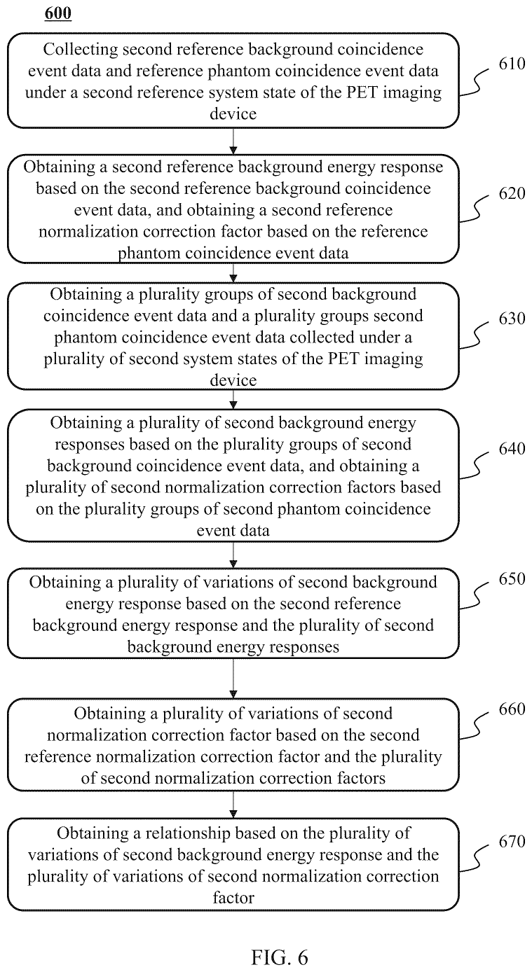

In some embodiments, the processing device 120 may obtain the background energy response and the normalization correction factor under different system states in the event of a change in the system state. The processing device 120 may compare the background energy response under different system states to obtain the variation of the background energy response under different system states, and compare the normalization correction factor under different energies to obtain the variation of the normalization correction factor. After obtaining the variation of the background energy response and the variation of the normalization correction factor under different system states, the processing device 120 may obtain, in a plurality of ways (e.g., curve fitting, etc.), a correlation between the two, i.e., a functional relationship between the variation of the background energy response and the variation of the normalization correction factor, which may be denoted as F. In some embodiments, the processing device 120 may experimentally obtain the functional relationship F. For example, the functional relationship between a variation of the crystal detection efficiency correction factor and a variation of the position of a 307 keV energy peak (i.e., the functional relationship between the variation of the background energy response and the variation of the normalization correction factor) may be obtained based on data of a uniform phantom and background radiation under different system states. More descriptions regarding obtaining the relationship may be found in FIG. 6 and related descriptions thereof.

The following illustration in the present disclosure uses the normalization correction factor as the crystal detection efficiency correction factor as an example only, which is not intended to be a limitation.

In some embodiments, the processing device 120 may obtain the functional relationship F between the variation of the background energy response and the variation of the normalization correction factor by various means (e.g., actual testing such as experiments, statistics based on historical data, etc.), and the functional relationship F may be represented by equation (9) as follows:

$$Δ ε = F [ P ( E ) , {P}^{′} ( E ) ] , \qquad ( 9 )$$

where Δε denotes the variation of the crystal detection efficiency correction factor; P(E) denotes a reference background energy response; and P′(E) denotes the background energy response after changing the system state.

In some embodiments, if the energy responses P(E) and P′(E) are assumed to conform to a Gaussian distribution, a Gaussian fit may be performed, as shown in equations (10) and (11) as follows:

$$P ( E ) = \frac{A}{\sqrt{2 π} σ} exp [ - \frac{{( E - μ )}^{2}}{2 {σ}^{2}} ] , \qquad ( 10 )$$

$${P}^{′} ( E ) = \frac{{A}^{′}}{\sqrt{2 π} {σ}^{′}} exp [ - \frac{{( E - {μ}^{′} )}^{2}}{2 {σ}^{′2}} ] , \qquad ( 11 )$$

where E denotes the background energy (e.g., 589 keV, 307 keV, etc.); A, μ, σ, and A′, μ′, σ′ denote Gaussian fitting parameters.

In some embodiments, based on equations (9)-(11), a relationship between Δε and P(E) and a relationship between Δε and P′(E) may be simplified to equation (12) as follows:

$$Δ ε = F [ A , μ , σ , {A}^{′} , {μ}^{′} , {σ}^{′} ] . \qquad ( 12 )$$

In some embodiments, assuming Δε=F[μ, μ′], equation (12) may be further simplified to equations (13) and (14) as follows:

$$Δ ε = F [ Δμ ] , \qquad ( 13 )$$

$$Δμ = {μ}^{′} - μ . \qquad ( 14 )$$

In some embodiments, after obtaining the relationship between the variation of the background energy response and the variation of the normalization correction factor, the processing device 120 may apply the relationship to the normalization correction of clinical scan data to correct a scanned image.

In 510, first background coincidence event data of a detector crystal of a PET imaging device is acquired at a predetermined time point.

In some embodiments, the processing device 120 may acquire background radiation data of the detector crystal of the PET imaging device at the predetermined time point as the first background coincidence event data. In some embodiments, the predetermined time point may be periodic. For example, the period is any of 24 hours, 48 hours, 72 hours, etc. In some embodiments, the predetermined time point may be determined based on a count of device works and/or a work duration. For example, the device works 5 times, 10 times, 15 times, etc. For example, the device works for 4 hours, 8 hours, 12 hours, etc. Because the system state changes over time, in some embodiments, the background coincidence event data of the detector crystal acquired at the predetermined time point may include the background radiation data after changing the system state, and the change in the system state is relative to a first reference system state.

The reference system state refers to a system state that serves as a reference for comparison with other system states after the change in the system state and may be a system state when the system operates normally for a long period of time. There may be a plurality of reference system states (e.g., a first reference system state, a second reference system state, etc.), and these reference system states may be the same or different. The first reference system state refers to a system state that is set as a reference for the purpose of normalizing and correcting the clinical scan data. In some embodiments, the first reference system state may be any of the selected system states. In some embodiments, the first reference system state may include a system state after a single normalization correction is performed.

In 520, the first background energy response is obtained based on the first background coincidence event data.

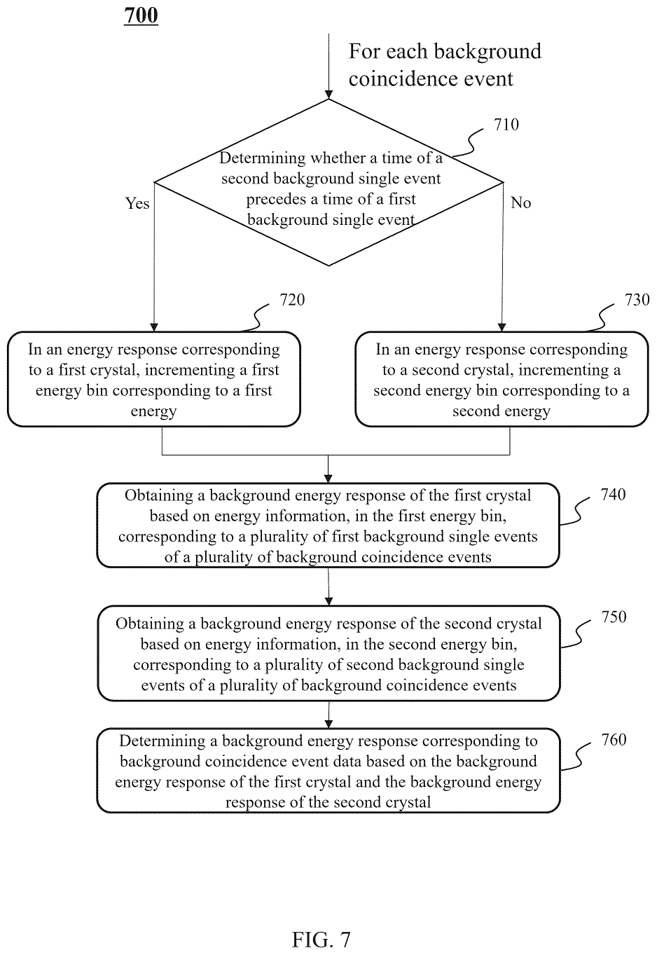

In some embodiments, the processing device 120 may obtain an energy response (e.g., the background energy response) by counting obtained single coincidence events (which may be referred to as a single event). The processing device 120 may obtain, by counting the single coincidence events in the first background coincidence event data, the background energy response and designate the obtained background energy response as the first background energy response, which is denoted as P′(E). More descriptions regarding obtaining the energy response may be found in FIG. 7 and related descriptions thereof.

In 530, the variation of the first background energy response is generated based on the first background energy response and the first reference background energy response.

In some embodiments, the processing device 120 may obtain the background energy response under the first reference system state of the PET imaging device as the first reference background energy response, which is denoted as P(E); and obtain the normalization correction factor by performing scanning the phantom and designate the normalization correction factor as a first reference normalization correction factor, which is denoted as NO. A manner for obtaining the normalization correction factor based on coincidence event data of the phantom is not limited to the present disclosure.

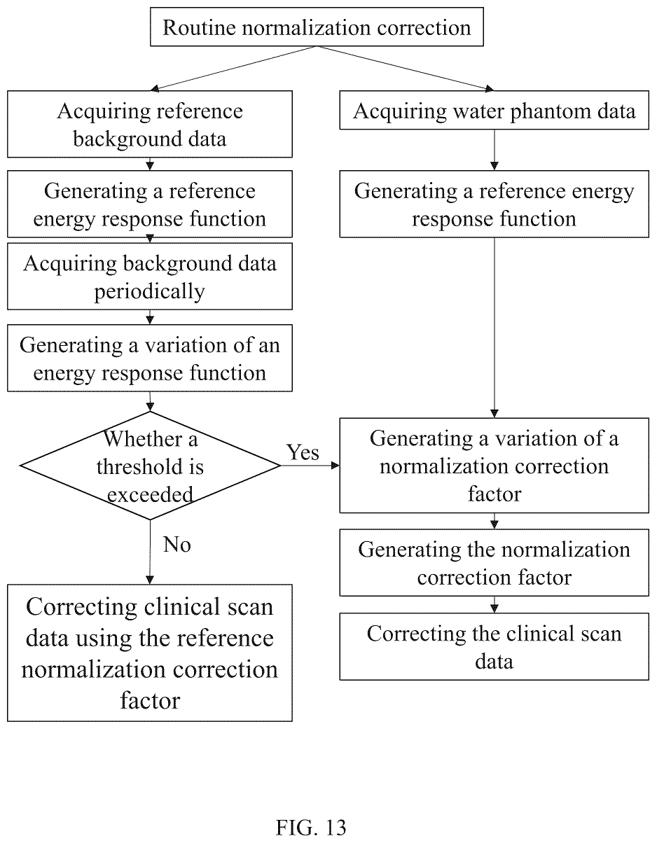

FIG. 13 is a schematic diagram illustrating an exemplary process of performing normalization correction on clinical scan data according to some embodiments of the present disclosure. As shown in FIG. 13, after the scanning system performs a routine normalization correction, the scanning system is under the first reference system state, then the processing device 120 may acquire the reference background data (i.e., the first reference background energy response) to generate a reference energy response function (e.g., an energy response function corresponding to 1130 in FIG. 11A) that represents the first reference background energy response, and acquire phantom (e.g., water phantom) data to generate the reference normalization correction factor (i.e., the first reference normalization correction factor), which is denoted as N₀.

In some embodiments, based on the first background energy response P′(E) and the reference background energy response P(E), the processing device 120 may generate the variation ΔP(E) of the first background energy response, which may be represented by the following equation (15):

$$Δ P ( E ) = {P}^{′} ( E ) - P ( E ) . \qquad ( 15 )$$

As shown in FIG. 13, after generating the reference energy response function, the processing device 120 may periodically acquire the background coincidence event data (i.e., the first background coincidence event data). After each acquisition, the processing device 120 may generate the variation of the energy response function (i.e., the variation ΔP(E) of the first background energy response) based on the background coincidence event data for this acquisition.

In some embodiments, after generating the variation of the first background energy response, the processing device 120 may determine whether the variation of the first background energy response is greater than a predetermined variation threshold. In response to determining that the variation of the first background energy response is not greater than the variation threshold, operation 540 is performed. In response to determining that the variation of the first background energy response is greater than the variation threshold, operations 550 and 560 are performed. In some embodiments, the variation threshold may be determined in various ways. For example, the variation threshold may be an empirical value or may be obtained based on historical data, etc.

In some embodiments, the variation threshold may be negatively correlated with an effect threshold of a target registration parameter in operation 440. For example, the larger the effect threshold, the smaller the variation threshold. The higher the effect threshold, the higher the requirements for registration, and the higher the requirements for the quality of the PET image. The higher the variation threshold, the longer the update cycle is indicated, which will lead to a gradual expansion of the mismatch between the normalization correction factor and the system state, making it difficult to ensure the effectiveness of the normalization correction, resulting in the degradation of the quality of the clinical scan images. By setting the variation threshold to be negatively correlated with the effect threshold, a matching degree of the normalization correction factor may be ensured, and the effect of the normalization correction may be guaranteed.

In 540, in response to determining that the variation of the first background energy response is not greater than the variation threshold, the first reference normalization correction factor is designated as the target normalization correction factor.

In some embodiments, if the variation of the first background energy response is not greater than the variation threshold, the processing device 120 may designate the first reference normalization correction factor as the target normalization correction factor. The target normalization correction factor is a normalization correction factor corresponding to a current system state of the PET imaging device (i.e., the system state of the PET imaging device corresponding to the predetermined time point in operation 510).

The present disclosure provides a manner for determining the target normalization correction factor, the variation under the current system state is determined to be less variable compared to the variation under the reference system state based on the fact that the variation of the background energy response is not greater than the variation threshold, so that the normalization correction factor under the reference system state is designated as the normalization correction factor under the current system state. The manner avoids frequent operations for determining the normalization correction factor, reduces ineffective workload, and obtains the normalization correction factor without requiring the radiation source and the CT imaging device. This simplifies the process of obtaining the normalization correction factor and reduces the requirements for operators and devices. The manner reduces the radiation damage to the operator from the radiation source, as well as the radiation damage to the patient from the CT imaging device and the external radiation source in the clinic. In addition, the method may be automatically performed outside of clinical scanning time (e.g., during non-working hours such as midnight), ensuring that normal clinical use is not interrupted.

In 550, in response to determining that the variation of the first background energy response is greater than the variation threshold, the variation of the first normalization correction factor is generated based on the variation of the first background energy response and a predetermined relationship.

In some embodiments, if the variation of the first background energy response is greater than the variation threshold, the processing device 120 may generate the variation of the first normalization correction factor (which is denoted as ΔN) based on the variation ΔP(E) of the first background energy response and the predetermined relationship. The predetermined relationship may include, for example, a functional relationship F between the variation of the background energy response and the variation of the normalization correction factor. For example, the predetermined relationship may be a functional relationship as described in FIG. 6. The variation ΔN of the first normalization correction factor may be expressed in equation (16) as follows:

$$Δ N = F [ P ( E ) , {P}^{′} ( E ) ] . \qquad ( 16 )$$

In some embodiments, the processing device 120 may obtain the variation of the first normalization correction factor in other ways. For example, the processing device 120 may obtain the variation of the first normalization correction factor via a machine learning model, a predetermined algorithm, etc. The processing device 120 may determine the variation of the first normalization correction factor based on the variation of the first background energy response through a correction prediction model, and the correction prediction model is a machine learning model.

In 560, the target normalization correction factor is determined based on the variation of the first normalization correction factor and the first reference normalization correction factor.

In some embodiments, the processing device 120 may correct the first reference normalization correction factor N₀ based on the variation ΔN of the first normalization correction factor, generate a new normalization correction factor under the current state, which is denoted as N₁, and designate the new normalization correction factor as the target normalization correction factor. In some embodiments, N₁ may be represented in equation (17) as follows:

$${N}_{1} = Δ N \times {N}_{0} . \qquad ( 17 )$$

In some embodiments, the processing device 120 may adjust the lower threshold of the energy window (e.g., 1110 in FIGS. 11A and 111B) at the predetermined time point, automatically or manually acquire the first background coincidence event data, perform the manner shown in operations 510-560, and correct a scanned image based on the target normalization correction factor. Specifically, after performing a clinical scan on a target object (e.g., a patient), the processing device 120 may correct the clinical scan data (i.e., the target coincidence event data of the target object) currently acquired using the target normalization correction factor, perform image reconstruction on the corrected clinical scan data based on an attenuation map, and obtain the scanned image. By redetermining the normalization correction factor, the accuracy of the normalization correction factor is improved, and the quality of a reconstructed image is enhanced. By adjusting the lower threshold of the energy window, the processing device 120 may acquire more background energy responses, thereby improving the accuracy of the redetermined normalization correction factor.

As shown in FIG. 13, after generating the variation of the energy response function, the processing device 120 may determine whether the variation exceeds the variation threshold. If the variation does not exceed the variation threshold, the processing device 120 may correct the clinical scan data using the reference normalization correction factor (as the target normalization correction factor). If the variation exceeds the variation threshold, the processing device 120 may correct the reference normalization correction factor based on the variation to generate the variation of the normalization correction factor. The processing device 120 may generate the normalization correction factor (as the target normalization correction factor) based on the variation of the normalization correction factor, and correct the clinical data based on the normalization correction factor.

In some embodiments of the present disclosure, by obtaining the relationship between the variation of the background energy response and the variation of the normalization correction factor, and correcting the normalization correction factor under the reference state using the relationship, to generate a new normalization correction factor. There is no need to re-calculate the normalization correction factor at background energy, no need to re-make the water phantom or use a solid source, no radiation source, no radiation damage, easy to operate, and may be performed outside of clinical scanning time (e.g., during non-working hours such as midnight), ensuring that the normal scanning time is not occupied, greatly improving the flexibility of time. The accuracy of the scanned data is ensured by correcting the clinical scan data using the new normalization correction factor, which improves and ensures the quality of the scanned reconstructed image.

FIG. 6 is a flowchart illustrating an exemplary process for determining a relationship between a variation of a background energy response and a variation of a normalization correction factor under different states of a PET imaging device according to some embodiments of the present disclosure. In some embodiments, at least part of the process 600 may be performed to achieve at least part of the operation as described in connection with FIG. 5. For example, the processing device 120 or the image reconstruction module 240 may determine a predetermined relationship by performing at least part of the process 600. The predetermined relationship includes the relationship between the variation of the background energy response and the variation of the normalization correction factor under different system states of the PET imaging device (e.g., the PET imaging device in the PET-CT system).

In 610, the second reference background coincidence event data and the reference phantom coincidence event data are collected under a second reference system state of the PET imaging device.

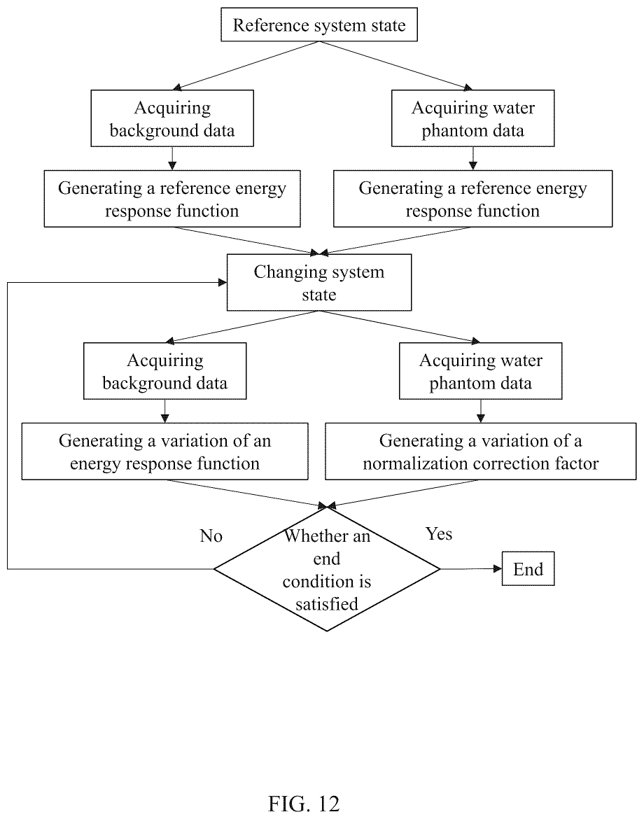

In some embodiments, the processing device 120 may collect background radiation data under the second reference system state as the second reference background coincidence event data, and collect the phantom coincidence event data of a phantom (e.g., a water phantom) as the reference phantom coincidence event data. The second reference state refers to a system state that is set as a reference in order to obtain the relationship between the variation of the background energy response and the variation of the normalization correction factor under different states of the scanning system. In some embodiments, the second reference state may be any one of the selected system states of the PET imaging device. In some embodiments, the second reference state may include a system state after one normalization correction. In some embodiments, the second reference state may be the same as or different from the first reference state. FIG. 12 is a schematic diagram illustrating an exemplary process for obtaining energy responses and normalization correction factors under different system states of a PET imaging device according to some embodiments of the present disclosure. As shown in FIG. 12, the processing device 120 may collect background data (i.e., background event data) and water phantom data under the reference system state.

In 620, a second reference background energy response is obtained based on the second reference background coincidence event data, and a second reference normalization correction factor is obtained based on the reference phantom coincidence event data.

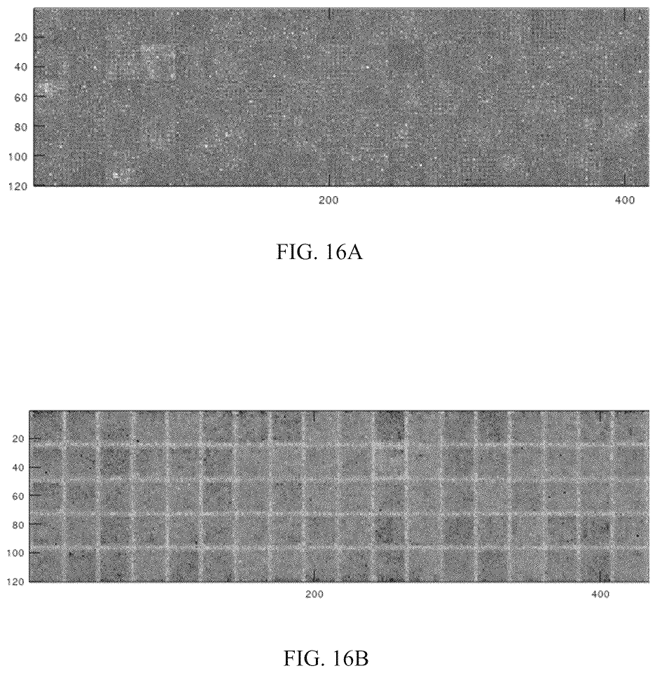

In some embodiments, the processing device 120 may obtain a background energy response based on the second reference background coincidence event data as the second reference background energy response, which is denoted as P₁(E). The crystal detection efficiency correction factor (denoted as ε) is illustrated below as an example of the normalization correction factor. In some embodiments, the processing device 120 may obtain, under the reference system state, using a peak-finding algorithm (e.g., Gaussian fitting, etc.), a position (denoted as μ₃₀₇) of a 307 keV energy peak of each crystal based on the background coincidence event data (e.g., the second reference background coincidence event data), which may be used as the second reference background energy response. The distribution of the position μ₃₀₇ of 307 keV energy peak may be as shown in FIG. 16B. In some embodiments, the processing device 120 may also designate other positions of the energy peak as a reference position of the energy peak. For example, any of 202 keV, 88 keV. More descriptions regarding obtaining the background energy response based on background coincidence event data may be found in FIG. 7.

In some embodiments, the processing device 120 may obtain the normalization correction factor (denoted as N₂) based on the reference phantom coincidence event data as the second reference normalization correction factor. The present disclosure does not limit the manner for obtaining the normalization correction factor based on the phantom coincidence event data.

As shown in FIG. 12, the processing device 120 may generate the reference energy response function (e.g., the second reference background energy response) based on the collected background data, and generate the reference normalization correction factor (e.g., the second reference normalization correction factor) based on the collected water phantom data.

The normalization correction factor as the crystal detection efficiency correction factor (denoted as ε) is still taken as an example for illustration. In some embodiments, the processing device 120 may, under a system state that serves as a reference, perform a normalization correction on a uniform water phantom to obtain the crystal detection efficiency correction factor ε, which may serve as the second reference normalization correction factor. The distribution of the crystal detection efficiency correction factor E may be as shown in FIG. 16A.

In 630, a plurality groups of second background coincidence event data and a plurality groups of second phantom coincidence event data collected under a plurality of second system states of the PET imaging device may be obtained.

In some embodiments, the processing device 120 may collect the background coincidence event data and the phantom coincidence event data after a change in the system state, i.e., under the second system state that is different from the second reference system state. In some embodiments, the processing device 120 may collect the plurality groups of second background coincidence event data and the plurality groups of second phantom coincidence event data collected in the plurality of second system states. The processing device 120 may collect the second background coincidence event data and the second phantom coincidence event data under a current second system state at each second system state, i.e., each time the system state changes. In some embodiments, the second system state may not be the same as the system state corresponding to the predetermined time point in operation 510.

In some embodiments, the change in the system state may be realized in various ways, such as by passive waiting or active control. The processing device 120 may change the system state by actively changing a crystal parameter, e.g., the processing device 120 may change a SiPM gain corresponding to a part of the crystal. The processing device 120 may change the system state by changing the environment in which a detector is located. For example, the processing device 120 may change the ambient temperature by changing a set temperature of a cooling system.

In some embodiments, the processing device 120 may obtain the relationship based on the plurality groups of second background coincidence event data and the plurality groups of second phantom coincidence event data by performing operations 640-670.

In 640, a plurality of second background energy responses are obtained based on the plurality groups of second background coincidence event data, and a plurality of second normalization correction factors are obtained based on the plurality groups of second phantom coincidence event data.

A background energy response is a response of the PET imaging device's detectors to specific energy γ photons, mainly affected by system changes such as aging and temperature changes. When the system changes occur, gain of the detector will change, and an energy spectrum output by the detectors will drift (also known as energy response change), resulting in distortion of the imaging of the detectors, and ultimately leading to a decrease in the spatial resolution of the detectors. For a specific normalization correction factor, it may be necessary to obtain a plurality of energy response of a plurality of crystals in different system states. For example, for crystal detection efficiency correction factor and axial profile correction factor, it is necessary to obtain the plurality of energy response of the plurality of crystals in each system state of a plurality of system states.

In some embodiments, under each second system state, the processing device 120 may obtain at least one second background energy responses relating to the second system state, denoted as

$${P}_{1}^{′} ( E ) ,$$

by obtaining a corresponding second background energy response based on the second background coincidence event data of the system state.

In some embodiments, under each second system state, the processing device 120 may obtain the second normalization correction factor (denoted as N3) corresponding to the second phantom coincidence event data based on the second phantom coincidence event data of the system state, thereby obtaining the plurality of second normalization correction factors.

In some embodiments, the processing device 120 may obtain the relationship based on the plurality of second background energy responses and the plurality of second normalization correction factors by performing operations 650-670.

In 650, a plurality of variations of second background energy response are obtained based on the second reference background energy response and the plurality of second background energy responses.

In some embodiments, in each second system state, the processing device 120 may obtain a variation of a second background energy response, denoted ΔP₁(E), based on a second background energy response

$${P}_{1}^{′} ( E )$$

and a second reference background energy response P₁(E), thereby obtaining a plurality of variations of second background energy response. A relationship between P₁(E),

$${P}_{1}^{′} ( E ) ,$$

and ΔP₁(E) may be shown in equation (18) as follows:

$$Δ {P}_{1} ( E ) = {P}_{1}^{′} ( E ) - {P}_{1} ( E ) , \qquad ( 18 )$$

equation (18) is similar to equation (15), wherein P₁(E),

$${P}_{1}^{′} ( E ) ,$$

and ΔP₁(E) are equivalent to P(E), P′(E), and ΔP(E) in equation (15), respectively.

In some embodiments, for each second system state, the processing device 120 may select a predetermined count of target crystals from a plurality of detector crystals in the PET imaging device. For each target crystal, the processing device 120 may determine, based on a second reference background energy response corresponding to the target crystal and a second background energy response corresponding to the target crystal, variations of the background energy response of the target crystal under the second system state and the second reference system state. The processing device 120 may weight and sum the variations of the background energy response corresponding to the predetermined count of target crystals to obtain the variation of the background energy response under the second system state.

In some embodiments, the weight corresponding to the target crystal may be positively correlated with the variation of the background energy response corresponding to the target crystal. For example, the greater the variation of the background energy response corresponding to the target crystal, the greater the corresponding weight.

In some embodiments, the target crystal may correspond to at least one target energy. For each target energy of the at least one target energy, the processing device 120 may determine, based on the second reference background energy response of the target crystal to the target energy under the second reference system state and the second background energy response of the target crystal to the target energy under the second system state, the variations of the background energy response of the target crystal to the target energy under the second system state and the second reference system state. The processing device 120 may weight and sum the variation of the background energy response corresponding to the at least one target energy to obtain the variations of the background energy response of the target crystal to the target energy under the second system state and the second reference system state.

In 660, a plurality of variations of second normalization correction factor are obtained based on the second reference normalization correction factor and the plurality of second normalization correction factors.

In some embodiments, under each second system state, the processing device 120 may obtain a corresponding variation of a second normalization correction factor, denoted as ΔN₁, based on a second normalization correction factor N₃ and a second reference normalization correction factor N₂, thereby obtaining the plurality of variations of second normalization correction factor. In some embodiments, a relationship between N₂, N₃, and ΔN₁ is shown in equation (19) as follows:

$${N}_{3} = Δ {N}_{1} \times {N}_{2} , \qquad ( 19 )$$

equation (19) may be similar to equation (17), wherein N₂, N₃, and ΔN₁ are equivalent to N₀, N₁, and ΔN in equation (17), respectively.

From equation (19), the variation ΔN₁ of the second normalization correction factor may be shown in equation (20) as follows:

$$Δ {N}_{1} = {N}_{3} / {N}_{2} . \qquad ( 20 )$$

As shown in FIG. 12, after generating the reference energy response function (i.e., the second reference background energy response) and the reference normalization correction factor (i.e., the second reference normalization correction factor), the processing device 120 may change the system state a plurality of times. After each system state change, the processing device 120 may collect background data, generate a variation of the energy response function (e.g., the variation of the second background energy response) based on the background data, collect water phantom data, generate a variation of the normalization correction factor (e.g., the variation of the second normalization correction factor) based on the water phantom data, and then, determine whether an end condition has been reached. If the end condition is reached, the processing device 120 may stop changing the system state, i.e., stop acquiring the variation of the energy response function or the variation of the normalization correction factor. If the end condition is not reached, the processing device 120 may make next change in the system state, continuing to acquire the variation of the energy response function and the variation of the normalization correction factor. In some embodiments, the end condition may include various forms. For example, a count of the change in the system state has reached a predetermined threshold. As another example, a difference between variations of the energy response function or variations of the normalization correction factor in sequentially adjacent post-change systems, and the end condition may be that the difference is less than a threshold a plurality of consecutive times (e.g., ≥3 times), which indicate that the scanning system has stabilized and that there is little change in its parameter (e.g., the normalization correction factor).

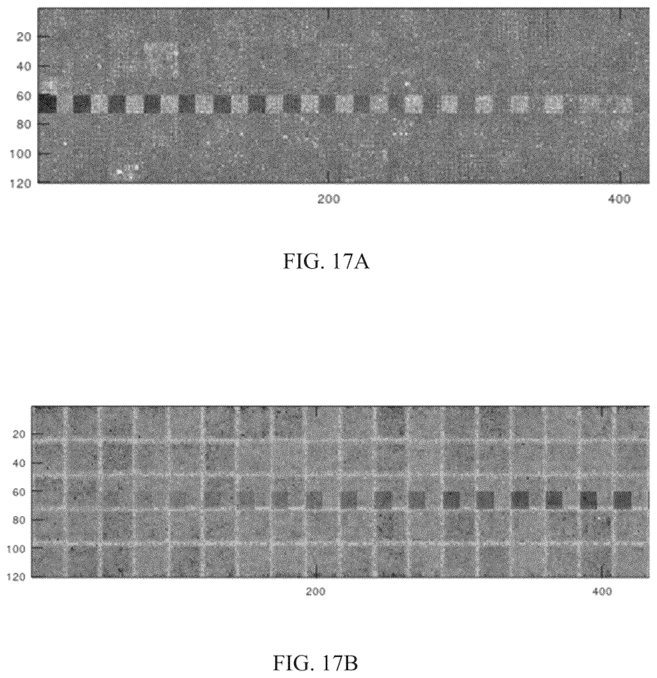

The normalization correction factor as the crystal detection efficiency correction factor (denoted as ε) is still used as an example. After obtaining the crystal detection efficiency correction factor ε and the position μ₃₀₇ of the 307 keV energy peak under the reference system state, the processing device 120 may change the system state and perform the normalization correction on the uniform water phantom again under the new system state, to obtain the crystal detection efficiency correction factor ε′, which may be designated as the second normalization correction factor. The distribution of the crystal detection efficiency correction factor ε′ may be as shown in FIG. 17A. The processing device 120 may collect the background coincidence event data (the second background coincidence event data) again under the new system state, and utilize a peak-finding algorithm (e.g., Gaussian fitting, etc.) to obtain the position of the 307 keV energy peak of each crystal, denoted as

$${μ}_{307}^{′} ,$$

which may serve as the second background energy response. The distribution of the position

$${μ}_{307}^{′}$$

of the 307 keV energy peak may be as shown in FIG. 17B.

After obtaining the crystal detection efficiency correction factor ε′ and the position

$${μ}_{307}^{′}$$

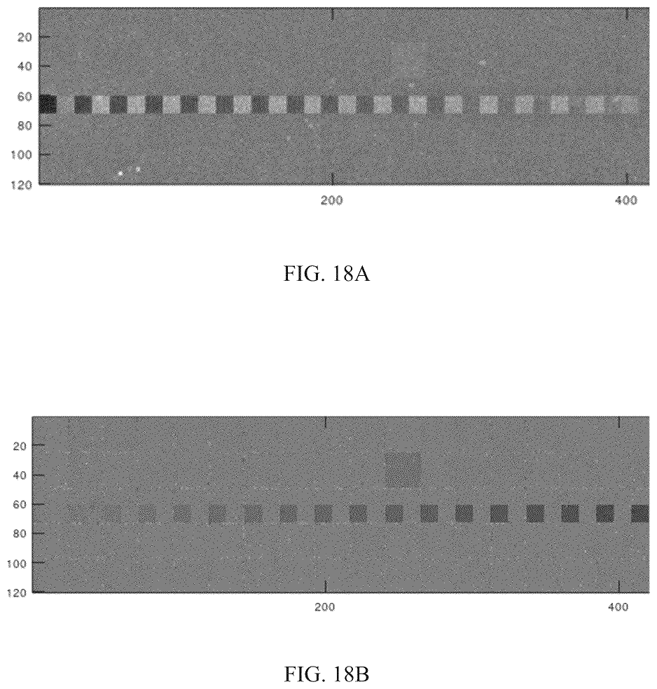

of the 307 keV energy peak, the processing device 120 may obtain the variation Δε of the crystal detection efficiency correction factor according to equation (20) based on the crystal detection efficiency correction factors ε and ε′ obtained as described previously, where Δε=ε/ε′. The distribution of the variation Δε of the crystal detection efficiency correction factor may be as shown in FIG. 18A. The processing device 120 may obtain a variation Δμ₃₀₇ of the position of the 307 keV energy peak based on the positions μ₃₀₇ and

$${μ}_{307}^{′}$$

of the 307 keV energy peak obtained as previously described, according to equation (18), where

$$Δ {μ}_{307} = {μ}_{307}^{′} - {μ}_{307} .$$

The distribution of the variation Δμ₃₀₇ of the position of the 307 keV energy peak may be as shown in FIG. 18 B.

In 670, a functional relationship is obtained based on the plurality of variations of second background energy response and the plurality of variations of second normalization correction factor.

In some embodiments, the processing device 120 may obtain a functional relationship F between the variation of background energy response and the variation of normalization correction factor, based on a plurality of variations ΔP₁(E) of second background energy response and a plurality of variations ΔN₁ of the second normalization correction factor obtained by curve fitting. In some embodiments, based on equation (10), the functional relationship F may be as shown in the equation (21) as follows:

$$Δ {N}_{1} = F [ {P}_{1} ( E ) , {P}_{1}^{′} ( E ) ] \qquad ( 21 )$$

equation (10) is similar to equation (1), where ΔN₁, P₁(E), and

$${P}_{1}^{′} ( E )$$

are equivalent to Δε, P(E), and P′(E) in equation (1), respectively.

The normalization correction factor as the crystal detection efficiency correction factor (denoted as ε) is still used as an example for illustration. In some embodiments, after obtaining a plurality of variations Δε of crystal detection efficiency correction factor and a plurality of variations of the position Δμ₃₀₇ of the 307 keV energy peak, the processing device 120 may utilize the plurality of variations Δε of crystal detection efficiency correction factor and the plurality of variations of the position Δμ₃₀₇ of the 307 keV energy peak position to perform a curve-fitting, to obtain the functional relationship F shown in equation (1). Curve fitting results of the variation of the crystal detection efficiency correction factor and the variation of the position of the 307 keV energy peak may be as shown in FIG. 17A and FIG. 17B.

In some embodiments, for normalization correction factors except the crystal detection efficiency correction factor (e.g., one or more of an axial profile correction factor, a detector ring pair detection efficiency correction factor, an annular profile correction factor, and a time-of-flight correction factor), the functional relationship F between the variation of the normalization correction factor and the variation of the energy response may be obtained using a manner similar to that used for obtaining the crystal detection efficiency correction factor.

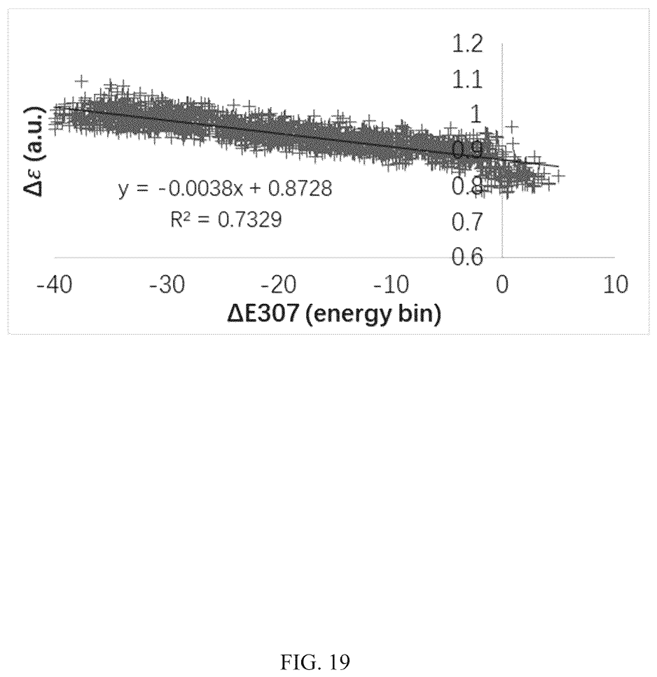

In some embodiments, the processing device 120 may obtain the functional relationship F between the variation of the normalization correction factor and the variation of the energy response in other ways, for example, through a machine learning model, etc. In some embodiments, the processing device 120 may input the background radiation data and the normalization correction factor (or the plurality variations of background energy response and the plurality variations of normalization correction factor) acquired under different system states into the machine learning model to obtain the functional relationship F. For example, the functional relationship F may be a relationship between the variation Δε of the crystal detection efficiency correction factor and the variation of the position of the 307 keV energy peak as shown in FIG. 19.

In some embodiments, the processing device 120 may obtain the relationship between the normalized correction factor after the system state change (e.g., the second normalized correction factor) and the background energy response after the system state change (e.g., the second background energy response) as the predetermined relationship. For example, the functional relationship G between the second normalized correction facto and the second background energy response may be as shown in the equation (22) as follows:

$${N}_{3} = G [ {P}_{1}^{′} ( E ) ] , \qquad ( 22 )$$

where N₃ denotes the second normalization correction factor,

$${P}_{1}^{′} ( E )$$

denotes the second background energy response. The equation (22) may be obtained by deforming equations (20) and (21), where the second reference normalization correction factor N₂ in the equation (20) and the second reference background energy response P₁(E) in the equation (21) are assumed to be known values.

In some embodiments of the present disclosure, by counting the variation of the background energy response and the variation of the normalization correction factor under different system states, and the functional relationship between the variation of normalization correction factor and the variation of energy response by curve fitting. Thus, the normalization correction factor under the current system state may be accurately determined based on the variation of the energy response using the functional relationship. Improving the accuracy of the normalization correction factor overcomes the problem that the mismatch between the normalization correction factor and the system state gradually expands over time, thereby improving the quality of the clinical scanned image.

FIG. 7 is a flowchart illustrating an exemplary process for obtaining a background energy response based on background coincidence event data according to some embodiments of the present disclosure. In some embodiments, at least part of the process 700 may be performed to achieve at least part of operation 520 as described in connection with FIG. 5. For example, the processing device 120 or the image reconstruction module 240 may obtain the background energy response based on the background coincidence event data by performing at least part of the process 700.

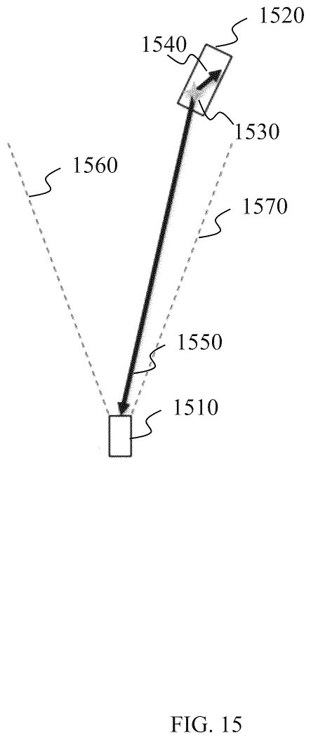

The background radiation is typically weak and not easily detectable. Because gamma decay has a specific energy, in some embodiments, for a background coincidence event, the processing device 120 may utilize gamma decay, i.e., designate the received gamma decay as a single event. In order to improve the statistical quality of the background coincidence event, the processing device 120 may take a crystal as a pyramidal tract at the top, count the background coincidence event within the pyramidal tract, accumulate energy information of all coincidence events with the crystal as an end, to obtain an energy response of the crystal. As shown in FIGS. 15, 1510 and 1520 are different crystals, 1530 is a position where Lu-176 decays, the upward arrow 1540 indicates beta decay, the downward arrow 1550 indicates the gamma decay, and two dashed lines 1560 and 1570 indicate boundaries of the pyramidal tract. From FIG. 15, it may be seen that the gamma decay has a longer flight path than the beta decay, making a flight time longer, and therefore, the single event occurs later. In some embodiments, the processing device 120 may determine energy of the single event based on an arrival time, i.e., a single event with a later arrival time is determined to be the gamma decay. As shown in FIG. 15, the processing device 120 may accumulate the energy information of all coincidence events with the crystal 1510 as an end, to obtain an energy response of the crystal 1510.

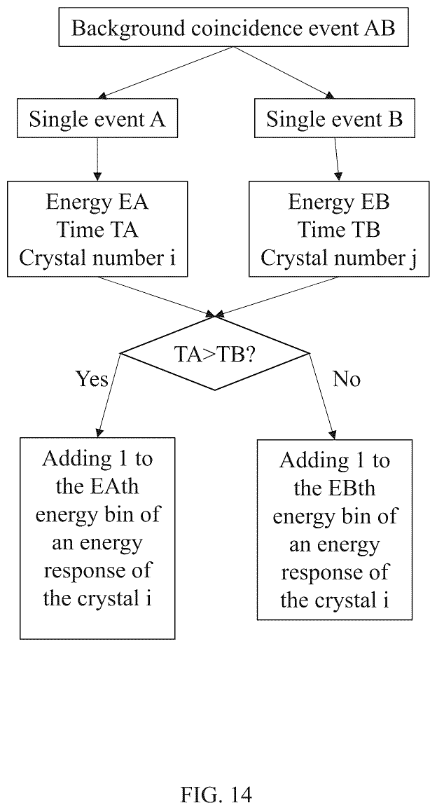

In some embodiments, each coincidence event of a plurality of coincidence events corresponding to the coincidence event data includes a first single event and a second single event, and the processing device 120 may obtain the first single event and the second single event from the obtained coincidence event data (e.g., the first reference coincidence event data, the second reference coincidence event data, the reference phantom coincidence event data). The first background single event corresponds to first energy and a first crystal, and the second background single event corresponds to second energy and a second crystal. The first energy and the second energy correspond to different energy decays, i.e., the first energy is energy corresponding to β rays or γ rays related to the first crystal, and the second energy is energy corresponding to the γ rays or β rays related to the second crystal. For example, if the first energy is gamma decay, the second energy is beta decay. For example, if the first energy is beta decay, the second energy is gamma decay. The first background single event and the second background single event are not generated on a same crystal. For example, the first background single event is generated on the first crystal and the second background single event is generated on the second crystal. The first crystal and the second crystal are located on different detectors. For example, the first crystal is located on detector A and the second crystal is located on detector B. FIG. 14 is a schematic diagram illustrating an exemplary process for obtaining a background energy response according to some embodiments of the present disclosure. As shown in FIG. 14, the processing device 120 may obtain a single event A (i.e., the first background single event) and a single event B (i.e., the second background single event) from a background coincidence event AB. The single event A has energy EA, time TA, and crystal number i; and the single event B has energy EB, time TB, and crystal number j.

In some embodiments, the processing device 120 may determine the background energy response by performing operations 710-770 for a plurality of background coincidence events corresponding to the background coincidence event data.

In 710, whether a time of the second background single event precedes a time of the first background single event is determined.

In some embodiments, for each of the plurality of background coincidence events corresponding to the background coincidence event data, the processing device 120 may determine whether the time of the second background single event precedes the time of the first background single event. If the time of the second coincidence single event precedes the time of the first coincidence single event, operation 720 is performed, otherwise, operation 730 is performed. As shown in FIG. 14, the processing device 120 may determine whether TA is greater than TB, and perform different operations according to the determination result.

In 720, in response to determining that the time of the second background single event precedes the time of the first background single event, in an energy response corresponding to the first crystal, a first energy bin corresponding to the first energy is incremented.

The energy bin refers to an energy range used to determine the energy response, which may be determined from an energy level of the decay (e.g., beta decay, gamma decay, etc.). For example, the energy bin may be a range centered on any of 589 keV, 307 keV, 202 keV, and 88 keV, etc. In some embodiments, an energy range of the energy bin may be smaller than an energy range of an energy window.

In some embodiments, if the time of the second background single event precedes the time of the first background single event, the processing device 120 may increment, in the energy response corresponding to the first crystal, the first energy bin corresponding to the first energy. The first energy bin refers to an energy range of the first energy. Merely by way of example, if the time of the second background single event precedes the time of the first background single event, the processing device 120 may determine that the first energy is gamma decay, i.e., the first crystal receives a gamma decay event, and the processing device 120 may add 1 to the first energy bin corresponding to the first energy of the first crystal, i.e., add 1 to a count of the first energy in the first energy bin. As shown in FIG. 14, if TA is greater than TB, i.e., the time of the second background single event precedes the time of the first background single event, then the processing device 120 may determine that EA is gamma decay, and add 1 to the EAth energy bin of an energy response of the crystal i.

Step 730, in response to determining that the time of the second background single event does not precede the time of the first background single event, in an energy response corresponding to the second crystal, a second energy bin corresponding to the second energy is incremented.

In some embodiments, if the time of the second background single event does not precede the time of the first background single event, the processing device 120 may increment, in the energy response corresponding to the second crystal, the second energy bin corresponding to the second energy. The second energy bin refers to an energy range for the second energy. Merely by way of example, if the time of the second background single event does not precede the time of the first background single event, the processing device 120 may determine that the second energy is gamma decay, i.e., the second crystal receives a gamma decay event, and the processing device 120 may add 1 to the second energy bin corresponding to the second energy of the second crystal, i.e., add 1 to a count of the second energy in the second energy bin. As shown in FIG. 14, if TA is less than or equal to TB, i.e., the time of the second background single event does not precede the time of the first background single event, then the processing device 120 may determine that EB is gamma decay and add 1 to the EBth energy bin of the energy response of the crystal j.

In some embodiments, after performing the operations 710-730 for all background coincidence events corresponding to the background coincidence event data, the processing device 120 may continue to perform operations 740-760 to obtain a background energy response corresponding to the background coincidence event data.

In 740, a background energy response of the first crystal is obtained based on energy information, in the first energy bin, corresponding to a plurality of first background single events of the plurality of background coincidence events.

In some embodiments, the processing device 120 may obtain the background energy response of the first crystal based on the energy information, i.e., the count of the first energy in the first energy bin, of the plurality of first background single events corresponding to the plurality of background coincidence events. It is to be understood that the count of the first energy in the first energy bin reflects a count of the background coincidence event from the first crystal.

In 750, a background energy response of the second crystal is obtained based on energy information, in the second energy bin, corresponding to a plurality of second background single events of the plurality of background coincidence events.

In some embodiments, the processing device 120 may obtain the background energy response of the second crystal based on the energy information, i.e., a count of the second energy in the second energy bin, of a plurality of second background single events corresponding to the plurality of background coincidence events. It may be appreciated that the count of the second energy in the second energy bin reflects a count of background coincidence events from the second crystal.

In 760, the background energy response corresponding to the background coincidence event data is determined based on the background energy response of the first crystal and the background energy response of the second crystal.

In some embodiments, the processing device 120 may sum the background energy response of the first crystal and the background energy response of the second crystal to obtain the background energy response corresponding to the background coincidence event data.

In some embodiments of the present disclosure, the statistical accuracy of the background coincidence event is improved by determining the background coincidence event based on the chronological order of the single events in gamma decay, and the statistical accuracy of the background coincidence event is improved by taking radiation data within the pyramidal tract with the crystal as a top.

The operations of the illustrated processes 300, 400, 500, 600, and 700 presented above are intended to be illustrative. In some embodiments, a process may be accomplished with one or more additional operations not described, and/or without one or more of the operations discussed. Additionally, the order in which the operations of a process described above is not intended to be limiting.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” may mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “unit,” “module,” or “system.” Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2103, Perl, COBOL 2102, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, for example, an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various inventive embodiments. This method of disclosure, however, is not to be interpreted as reflecting an intention that the claimed object matter requires more features than are expressly recited in each claim. Rather, inventive embodiments lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±1%, ±5%, ±10%, or ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1. A method, performed by a computing device including at least one processor and at least one storage device, wherein the method is implemented on a PET-CT system, the PET-CT system includes a computed tomography (CT) imaging device and a positron emission computed tomography (PET) imaging device, and the method comprises:
obtaining PET data of a target object within a field of view (FOV) of the PET imaging device;
obtaining CT data of the target object within a FOV of the CT imaging device;
generating an attenuation image based on a target registration parameter and the CT data, wherein the target registration parameter characterizes a mapping relationship registering the CT coordinate system and the PET coordinate system, and the target registration parameter is obtained based on background coincidence event data from the PET imaging device; and
reconstructing a target PET image based on the PET data and the attenuation image.

2. The method of claim 1, wherein the obtaining PET data comprises:
obtaining corrected PET data by correcting the PET data based on a target normalization correction factor, wherein the target normalization correction factor is a normalization correction factor corresponding to a current state of the PET imaging device, and the target normalization correction factor is determined by:
acquiring first background coincidence event data of a detector crystal of the PET imaging device at a predetermined time point; and
determining the target normalization correction factor based on the first background coincidence event data.

3. The method of claim 2, wherein the determining the target normalization correction factor based on the first background coincidence event data comprises:
obtaining a first background energy response based on the first background coincidence event data; and
determining the target normalization correction factor based on the first background energy response.

4. The method of claim 3, wherein the determining the target normalization correction factor based on the first background energy response comprises:
generating a variation of the first background energy response based on the first background energy response and a first reference background energy response, wherein the first reference background energy response is obtained under a first reference system state of the PET imaging device;
in response to determining that the variation of the first background energy response is not greater than a variation threshold, designating a first reference normalization correction factor as the target normalization correction factor, wherein the first reference normalization correction factor is obtained based on a phantom under the first reference system state of the PET imaging device; or
in response to determining that the variation of the first background energy response is greater than the variation threshold, generating a variation of a first normalization correction factor based on the variation of the first background energy response and a predetermined relationship; and
determining the target normalization correction factor based on the variation of the first normalization correction factor and the first reference normalization correction factor.

5. The method of claim 4, wherein the predetermined relationship includes a relationship between the variation of the background energy response and the variation of the normalization correction factor, and the relationship is obtained by:
collecting second reference background coincidence event data and reference phantom coincidence event data in a second reference system state of the PET imaging device;
obtaining a second reference background energy response based on the second reference background coincidence event data,
obtaining a second reference normalization correction factor based on the reference phantom coincidence event data;
obtaining a plurality groups of second background coincidence event data and a plurality groups of second phantom coincidence event data collected under a plurality of second system states of the PET imaging device, wherein each of the plurality of second system states is different from the second reference system state; and
obtaining the relationship based on the plurality groups of second background coincidence event data and the plurality groups of second phantom coincidence event data.

6. The method of claim 5, wherein the obtaining the relationship based on the plurality groups of second background coincidence event data and the plurality groups of second phantom coincidence event data comprises:
obtaining a plurality of second background energy responses based on the plurality groups of second background coincidence event data,
obtaining a plurality of second normalization correction factors based on the plurality groups of second phantom coincidence event data; and
obtaining the relationship based on the plurality of second background energy responses and the plurality of second normalization correction factors.

7. The method of claim 6, wherein the obtaining the relationship based on the plurality of second background energy responses and the plurality of second normalization correction factors comprises:
obtaining a plurality of variations of second background energy response based on the second reference background energy response and the plurality of second background energy responses;
obtaining a plurality of variations of second normalization correction factor based on the second reference normalization correction factor and the plurality of second normalization correction factors; and
obtaining the relationship based on the plurality of variations of second background energy response and the plurality of variations of second normalization correction factor.

8. The method of claim 2, wherein the normalization correction factor includes at least one of an axial profile correction factor, a detector ring pair detection efficiency correction factor, an annular profile correction factor, a crystal detection efficiency correction factor, or a time-of-flight correction factor.

9. The method of claim 3, obtaining a background energy response based on the background coincidence event data includes:
each of a plurality of background coincidence events corresponding to the background coincidence event data including a first background single event and a second background single event, the first background single event corresponding to first energy and a first crystal, the second background single event corresponding to second energy and a second crystal, and the first background single event and the second background single event being not generated on a same crystal;
for the background coincidence event,
determining whether a time of the second background single event precedes a time of the first background single event;
in response to determining that the time of the second background single event precedes the time of the first background single event, in an energy response corresponding to the first crystal, incrementing a first energy bin corresponding to the first energy, wherein the first energy bin is an energy range of the first energy;
obtaining a background energy response of the first crystal based on energy information, in the first energy bin, corresponding to a plurality of first background single events of the plurality of background coincidence events;
obtaining a background energy response of the second crystal based on energy information, in the second energy bin, corresponding to a plurality of second background single events of the plurality of background coincidence events; and
determining the background energy response corresponding to the background coincidence event data based on the background energy response of the first crystal and the background energy response of the second crystal.

10. The method of claim 9, further comprising:
obtaining the plurality of background coincidence events based on arrival energies and arrival times of β rays and γ rays in the background coincidence event data.

11. The method of claim 1, wherein the target registration parameter is obtained by:
obtaining a CT image of a phantom by placing the phantom in the FOV of the CT imaging device, the phantom not containing a radiation source;
collecting background coincidence event data of the phantom by placing the phantom in the FOV of the PET imaging device;
acquiring a first attenuation image in a coordinate system of the CT imaging device based on the CT image; and
determining the target registration parameter based on target background coincidence event data, and the first attenuation image, wherein the target background coincidence event data is obtained based on the background coincidence event data of the phantom.

12. The method of claim 11, wherein the target background coincidence event data is obtained by:
obtaining background coincidence event data after noise reduction by performing noise reduction on the background coincidence event data of the phantom, and
designating the background coincidence event data after noise reduction as the target background coincidence event data.

13. The method of claim 12, wherein the performing noise reduction on the background coincidence event data includes:
merging at least two target response lines in original response lines in the background coincidence event data of the phantom into one combined response line, and
obtaining the background coincidence event data after noise reduction based on background coincidence event data received by at least one combined response line.

14. The method of claim 12, wherein the performing noise reduction on the background coincidence event data includes:
obtaining the background coincidence event data after noise reduction by smoothing or using AI noise reduction algorithm for processing background coincidence event data received by original response lines in the background coincidence event data of the phantom.

15. The method of claim 11, wherein the determining the target registration parameter based on target background coincidence event data, and the first attenuation image includes:
iteratively updating, based on the target background coincidence event data, the first attenuation image, and a predetermined registration parameter, the predetermined registration parameter using a maximum likelihood estimation (MLE) function until a preset end condition is satisfied; and
designating the predetermined registration parameter corresponding to a maximum value of the maximum likelihood estimation function as the target registration parameter.

16-17. (canceled)

18. A method, performed by a computing device including at least one processor and at least one storage device, wherein the method comprises:
obtaining PET data of a target object within a field of view (FOV) of a positron emission computed tomography (PET) imaging device;
obtaining corrected PET data by correcting the PET data based on a target normalization correction factor, wherein the target normalization correction factor is a normalization correction factor corresponding to a current state of the PET imaging device, and the target normalization correction factor is determined by:
acquiring first background coincidence event data of a detector crystal of the PET imaging device at a predetermined time point;
determining the target normalization correction factor based on the first background coincidence event data; and
reconstructing a target PET image based on the corrected PET data.

19. The method of claim 18, wherein the determining the target normalization correction factor based on the first background coincidence event data comprises:
obtaining a first background energy response based on the first background coincidence event data; and
determining the target normalization correction factor based on the first background energy response.

20. The method of claim 19, wherein the determining the target normalization correction factor based on the first background energy response comprises:
generating a variation of the first background energy response based on the first background energy response and a first reference background energy response, wherein the first reference background energy response is obtained under a first reference system state of the PET imaging device;
in response to determining that the variation of the first background energy response is not greater than a variation threshold, designating a first reference normalization correction factor as the target normalization correction factor, wherein the first reference normalization correction factor is obtained based on a phantom under the first reference system state of the PET imaging device; or
in response to determining that the variation of the first background energy response is greater than the variation threshold, generating a variation of a first normalization correction factor based on the variation of the first background energy response and a predetermined relationship; and
determining the target normalization correction factor based on the variation of the first normalization correction factor and the first reference normalization correction factor.

21-26. (canceled)

27. The method of claim 18, wherein the method is implemented on a PET-CT system, the PET-CT system includes a computed tomography (CT) imaging device and the PET imaging device, and the reconstructing a target PET image based on the corrected PET data includes:
obtaining CT data of the target object within a FOV of the CT imaging device;
generating an attenuation image based on a target registration parameter and the CT data, wherein the target registration parameter characterizes a mapping relationship registering the CT coordinate system and the PET coordinate system, and the target registration parameter is obtained based on background coincidence event data from the PET imaging device; and
reconstructing a target PET image based on the corrected PET data and the attenuation image.

28-34. (canceled)

35. A method, performed by a computing device including at least one processor and at least one storage device, wherein the method is implemented on a PET-CT system, the PET-CT system includes a computed tomography (CT) imaging device and a positron emission computed tomography (PET) imaging device, and the method comprises:
obtaining a CT image of a phantom by placing the phantom in the FOV of the CT imaging device, the phantom not containing a radiation source;
collecting background coincidence event data of the phantom by placing the phantom in the FOV of the PET imaging device;
acquiring a first attenuation image in a coordinate system of the CT imaging device based on the CT image; and
determining a target registration parameter based on target background coincidence event data, and the first attenuation image, wherein the target background coincidence event data is obtained based on the background coincidence event data of the phantom.

36-37. (canceled)
